(19) United States
(12) Patent Application Publication
(10) Pub. No.: US 2013/0177579 A1
(43) Pub. Date: Jul. 11, 2013

(54) ANTI-TRANSFERRIN RECEPTOR ANTIBODIES AND METHODS USING SAME

(71) Applicant: BIOALLIANCE C.V., Amsterdam Zuidoost (NL)

(72) Inventors: Shih-Yao LIN, Taipei (TW); Leewen LIN, Taipei (TW); Yu-Ying TSAI, Taipei (TW); Chiu-Chen HUANG, Taipei (TW); Feng-Lin CHIANG, Taipei (TW); Yu-Chi HSIEH, New Taipei City (TW)

(73) Assignee: BIOALLIANCE C.V., Amsterdam Zuidoost (NL)

(21) Appl. No.: 13/734,056

(22) Filed: Jan. 4, 2013

Related U.S. Application Data
(60) Provisional application No. 61/584,125, filed on Jan. 6, 2012.

(51) Int. Cl. C07K 16/28

(52) U.S. Cl. CPC C07K 16/28
USPC 424/174.1; 530/389.7; 530/387.3; 530/391.7; 530/391.3; 435/320.1; 435/69.6; 435/7.23; 435/334

(57) ABSTRACT

The present disclosure relates to antibodies that recognize a carbohydrate on transferrin receptor expressed by nonhematopoietic tumor or cancer cells and uses thereof.

### CROSS-REFERENCE TO RELATED APPLICATIONS

This application claims the priority benefit of U.S. provisional application Ser. No. 61/584,125, filed Jan. 6, 2012, which is incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 606592000700SEQLIST.txt, date recorded: Jan. 2, 2013, size: 27 KB).

### TECHNICAL FIELD

The present disclosure relates to antibodies that recognize a carbohydrate on transferrin receptor expressed by nonhematopoietic tumor or cancer cells and uses thereof.

### BACKGROUND

Post-translational modification is chemical modification of a protein after its translation and is one of the late steps in gene expression for many proteins. More than 100 different types of post-translational modifications are known, such as attaching to it the other biochemically functional group (e.g., acetate, phosphate, various lipids and carbohydrate), changing the chemical nature of an amino acid (e.g., citrullination), or making structural changes (e.g., disulfide formation). Such post-translational modification extends the range of function of the protein (Alberts et al. (2002), Molecular Biology of the Cell, 4th ed., page 355; Smith et al. (2005), Marks' Basic Medical Biochemistry: a clinical approach, 2nd ed., page 85).

Not only does post-translational modification occur in normal cells, it also plays major roles in neoplastic transformation of cells. The neoplastic transformation of cells is accompanied by a dynamic change of the cellular signaling process, resulting in alternations in gene expression, activation of certain cellular signaling pathways, enhanced proliferation and dysregulation of cell division and of death, etc. Post-translational modifications play pivotal roles in all of these activities because it is the chemical modification of key regulatory or structural proteins that dictate the status of most physiological events within the cells (Krueger et al. (2006), Molecular & cellular Proteomics 5: 1799-1810).

Among the diverse realm of post-translational modification, altered carbohydrate profile on the cell surface is a property common to apparently all tumors. The outer glycocalyx found on epithelial and mesenchymal cells serves multiple roles over a broad scope of cell interactions with its microenvironment such as hygroscopic protection, external molecular buffering, adhesion to extracellular matrix and intercellular adhesion. These interactions dictate many aspects of the intracellular signaling event and thus the cellular behavior. Pronounced alterations in glycan profiles clearly contribute to the ability of a cell to detach from its normal tissue site and possibly adhere within another organ site (behavior of invasion and metastasis). For example, sialyl Lewis structures, which are often found in tumors, show a propensity to bind selectins and likely confer the metastatic properties of these cells (Fuster et al. (2003), Cancer Res. 63: 2775-2781).

The transferrin receptor (“TfR”), also known as CD71, is a cell membrane-associated glycoprotein pivotally involved in the cellular uptake of iron and in the regulation of cell growth (Daniels et al. (2006), Clin Immunol 121, 144-158). Diferric Transferrin binds the transferrin receptor, internalized in clathrin-coated pits through receptor-mediated endocytosis. The decrease in pH in the endosome facilitates a conformational change of Transferrin and its subsequent release of iron (Cheng et al. (2004), Cell 116, 565-576). The apo-transferrin/TfR complex return to the cell surface where apo-Transferrin is released. In contrast to receptors such as EGFR that are internalized only after interaction with its ligand (ligand-mediated internalization), Transferrin receptor is constitutively internalized independently of ligand binding (Watts (1985), J Cell Biol 100, 633-637; Taetle (1990), Exp. Hematol. 18: 360-365; Trowbridge et al. (1993), Annu. Rev. Cell Biol. 9: 129-161; Kurten (2003), Adv. Drug Delivery Rev. 55, 1405-1419).

Transferrin receptor is ubiquitously expressed on normal cells and its expression is increased on cells with a high proliferation rate such as cells of the basal epidermis and intestinal epithelium, or on cells that require large amounts of iron, such as placental trophoblasts (for iron delivery to the fetus) or maturing erythroid cells (for heme synthesis) (Gatter et al. (1983), J Clin Pathol 36: 539-545; Omary et al. (1980), Nature 286: 888-891; Sutherland et al. (1981), Proc. Natl. Acad. Sci. USA. 87, 4515-4519; Shindelman et al. (1981), Int. J. Cancer 27: 329-334). TfR is significantly up-regulated (up to 100 folds) in a variety of malignant cells such as breast cancer, adenocarcinoma of the lung, glioma, transitional cell carcinoma of bladder, chronic lymphocytic leukemia, non-Hodgkin's lymphoma and multiple myeloma (Daniels et al. (2006), Clin Immunol 121, 144-158; Omary et al. (1980), Nature 286: 888-891; Sutherland et al. (1981), Proc. Natl. Acad. Sci. USA. 87, 4515-4519; Shindelman et al. (1981), Int. J. Cancer 27: 329-334; Daniels et al. (2006), Clin Immunol 121: 159-176; Gomme et al. (2005), Drug Discov. Today 10: 267-273; Prost et al. (1998), Int. J. Oncol 13: 871-875; Shinohara et al. (2000), Int. J. Oncol 17: 643-651). This could be attributed to the increased need for iron as a cofactor of the ribonucleotide reductase enzyme involved in DNA synthesis of rapidly dividing cells. Furthermore, in many cases, increased expression of transferrin receptor correlates with tumor stage and is associated with poor prognosis.

Structurally, human TfR is a homodimeric type II transmembrane protein of 180 kD. The 90-kD Subunit (760 amino acids) has a short, NH₂-terminal cytoplasmic region (residues 1-61) which contains the internalization motif YTFR, a single transmembrane pass (residues 62-88), and a large extracellular portion (ectodomain, residues 89-760), which contains a binding site for the 80-kD Transferrin molecule (Daniels et al. (2006), Clin Immunol 121, 144-158; Cheng et al. (2004), Cell 116, 565-576; Lawrence et al. (1999), Science 286, 779-782). The ectodomain contains 3 N-linked glycosylation sites and one O-linked glycosylation site. Glycosylation at these sites is required for adequate function of the receptor (Daniels et al. (2006), Clin Immunol 121, 144-158; Enns et al. (1981), Proc. Natl. Acad. Sci. USA. 778, 4222-4225; Hayes et al. (1994), Glycobiology 5, 227-232).

TfR has been explored as a target to deliver therapeutics into cancer cells due to its increased expression on malignant cells (up to 100-fold higher than the average expression of normal cells), accessibility on the cell surface, and constitutive endocytosis (Daniels et al. (2006), Clin Immunol 121, 144-158). TfR can be targeted by direct interaction with conjugates of its ligand transferrin (Tf) or by monoclonal antibodies specific for the TfR. Chemotherapeutic drug such as doxorubicin, cisplatin, chlorambucil, mytomycin, gemcitabine and daunorubicin, toxic proteins such as ricin, Saporin, diphtheria exotoxin, CRM 107 and bovine RNase, polymers/polyplexes, liposomes and nanoparticles have been conjugated directly to Tf for TfR targeting. Monoclonal antibodies anti-TfR have also been developed as the targeting agent to deliver chemotherapeutic drugs such as doxorubicin, plant toxins such as Ricin, saporin, gelonin, pokeweed, Luffa toxoin, fungal toxins, Pseudomonas exotoxin, Diphtheria exotoxin, angiogenin, Ribonuclease and siRNA into cells which showed cytotoxic effects including growth inhibition and/or induction of apoptosis in a variety of malignancies in vitro and in vivo including in patients (Daniels et al. (2006), Clin Immunol 121: 159-176; Qian et al. (2002), Pharmacol Rev. 64, 561-587).

The transferrin receptor is an attractive targeting molecule that has been used to treat a variety of malignancies. However, due to its ubiquitous expression in normal cells, safety is a concern in patient treatment. There is thus a need to develop safe cancer therapies targeting transferrin receptor.

All references cited herein, including patent applications and publications, are incorporated by reference in their entirety.

### SUMMARY OF THE INVENTION

The present invention aims at targeting cancer specific modifications on the transferrin receptors which occur only in cells with malignant transformation. Compared to targeting transferrin receptor protein itself, targeting cancer-specific modifications of transferrin receptors offers another level of safety protection since only malignant cells are targeted. The present disclosure provides antibodies that are specifically against modifications of transferrin receptors since the antibodies bind to transferrin receptor expressed by mammalian cancer cells but do not bind to transferrin receptor expressed by E. coli. The epitopes of these antibodies are cancer specific since these antibodies can bind to cancer cells such as pancreatic cancer cells, gastric cancer cells, colorectal cancer cells, lung cancer cells, ovarian cancer cells, prostate cancer cells, endometrial cancer cells, breast cancer cells, and liver cancer cells, but not normal cells including activated T cells (which express high levels of transferrin receptors), red blood cells (“RBC”), platelet, polymorphonuclear leucocytes (“PMN”), peripheral blood mononuclear cells (“PBMC”), and human umbilical vein endothelial cells (“HUVEC”). Moreover, the anti-TfR antibodies described herein have cytotoxicity functions and can induce either apoptosis or complement-dependent cytotoxicity (“CDC”) when incubated with cells expressing target molecules. The antibodies provided in the present disclosure may be used for anti-cancer treatment.

Provided herein are antibodies (such as isolated antibodies) that specifically bind to a modification (such as a carbohydrate) on a transferrin receptor (such as human transferrin receptor) expressed by cancer cells such as nonhematopoietic cancer cells but does not specifically bind to a transferrin receptor expressed by activated T cells or by Jurkat cells. In some embodiments, the binding of the antibody to the transferrin receptor is not inhibited by a carbohydrate comprising a Le^{a} structure. In some embodiments, the antibody is a monoclonal antibody. In some embodiments, the antibody binds to an epitope comprising a fucose moiety. In some embodiments, the antibody binds to an epitope comprising a sialyl moiety. In some embodiments, the antibody binds to an epitope not comprising a sialyl moiety. In some embodiments, the binding of the antibody to the transferrin receptor is not inhibited by a carbohydrate comprising a Le^{b}, Le^{y}, or Le^{x} structure.

In some embodiments, the antibody competes for binding to the transferrin receptor with an antibody comprising a heavy chain variable region comprising the three complementarity determining regions (“CDRs”) from (or of) SEQ ID NO:1 and/or a light chain variable region comprising the three CDRs from (or of) SEQ ID NO:3. In some embodiments, the antibody comprises a heavy chain variable region comprising the three CDRs from (or of) SEQ ID NO:1 and/or a light chain variable region comprising the three CDRs from (or of) SEQ ID NO:3. In some embodiments, the antibody comprises (i) a heavy chain variable region comprising a sequence at least about 95% identical to amino acids 20-138 of SEQ ID NO:1 and/or (ii) a light chain variable region comprising a sequence at least about 95% identical to amino acids 20-132 of SEQ ID NO:3. In some embodiments, the antibody comprises a heavy chain variable region comprising amino acids 20-138 of SEQ ID NO:1 and/or a light chain variable region comprising amino acids 20-132 of SEQ ID NO:3.

In some embodiments, the antibody competes for binding to the transferrin receptor with an antibody comprising a heavy chain variable region comprising the three CDRs from (or of) SEQ ID NO:5 and/or a light chain variable region comprising the three CDRs from (or of) SEQ ID NO:7. In some embodiments, the antibody comprises a heavy chain variable region comprising the three CDRs from (or of) SEQ ID NO:5 and/or a light chain variable region comprising the three CDRs from (or of) SEQ ID NO:7. In some embodiments, the antibody comprises (i) a heavy chain variable region comprising a sequence at least about 95% identical to amino acids 20-138 of SEQ ID NO:5 and/or (ii) a light chain variable region comprising a sequence at least about 95% identical to amino acids 21-128 of SEQ ID NO:7. In some embodiments, the antibody comprises a heavy chain variable region comprising amino acids 20-138 of SEQ ID NO:5 and/or a light chain variable region comprising amino acids 21-128 of SEQ ID NO:7.

In some embodiments, the antibody competes for binding to the transferrin receptor with an antibody comprising a heavy chain variable region comprising the three CDRs from (or of) SEQ ID NO:9 and/or a light chain variable region comprising the three CDRs from (or of) SEQ ID NO:11. In some embodiments, the antibody comprises a heavy chain variable region comprising the three CDRs from (or of) SEQ ID NO:9 and/or a light chain variable region comprising the three CDRs from (or of) SEQ ID NO:11. In some embodiments, the antibody comprises (i) a heavy chain variable region comprising a sequence at least about 95% identical to amino acids 20-136 of SEQ ID NO:9 and/or (ii) a light chain variable region comprising a sequence at least about 95% identical to amino acids 21-134 of SEQ ID NO:11. In some embodiments, the antibody comprises a heavy chain variable region comprising amino acids 20-136 of SEQ ID NO:9 and/or a light chain variable region comprising amino acids 21-134 of SEQ ID NO:11.

In some embodiments, the antibody competes for binding to the transferrin receptor with an antibody comprising a heavy chain variable region comprising the three CDRs from (or of) SEQ ID NO:13 and/or a light chain variable region comprising the three CDRs from (or of) SEQ ID NO:15. In some embodiments, the antibody comprises a heavy chain variable region comprising the three CDRs from (or of) SEQ ID NO:13 and/or a light chain variable region comprising the three CDRs from (or of) SEQ ID NO:15. In some embodiments, the antibody comprises (i) a heavy chain variable region comprising a sequence at least about 95% identical to amino acids 20-138 of SEQ ID NO:13 and/or (ii) a light chain variable region comprising a sequence at least about 95% identical to amino acids 23-130 of SEQ ID NO:15. In some embodiments, the antibody comprises a heavy chain variable region comprising amino acids 20-138 of SEQ ID NO:13 and/or a light chain variable region comprising amino acids 23-130 of SEQ ID NO:15.

In some embodiments, the antibody competes for binding to the transferrin receptor with an antibody comprising a heavy chain variable region comprising the three CDRs from (or of) SEQ ID NO:17 and/or a light chain variable region comprising the three CDRs from (or of) SEQ ID NO:18. In some embodiments, the antibody comprises a heavy chain variable region comprising the three CDRs from (or of) SEQ ID NO:17 and/or a light chain variable region comprising the three CDRs from (or of) SEQ ID NO:18. In some embodiments, the antibody comprises (i) a heavy chain variable region comprising a sequence at least about 95% identical to amino acids 1-119 of SEQ ID NO:17 and/or (ii) a light chain variable region comprising a sequence at least about 95% identical to amino acids 1-108 of SEQ ID NO:18. In some embodiments, the antibody comprises a heavy chain variable region comprising amino acids 1-119 of SEQ ID NO:17 and/or a light chain variable region comprising amino acids 1-108 of SEQ ID NO:18.

In some embodiments, the antibody is a humanized antibody. In some embodiments, the antibody is a chimeric antibody. In some embodiments, the antibody is a human antibody. In some embodiments, the antibody is IgG (such as IgG₁, IgG₂, or IgG₄). In some embodiments, the antibody is human IgG such as human IgG₁.

In some embodiments, the antibody specifically binds to a transferrin receptor expressed by nonhematopoietic cancer cells such as pancreatic cancer cells, gastric cancer cells, colorectal cancer cells, lung cancer cells, ovarian cancer cells, prostate cancer cells, endometrial cancer cells, breast cancer cells, or liver cancer cells. In some embodiments, the antibody does not bind to a transferrin receptor expressed by CHO cells, red blood cells, platelets, HUVEC cells, monocytes, PMN, T cells, or activated T cells. In some embodiments, the cancer cells are human cancer cells. In some embodiments, the antibody is internalized (e.g., internalized in cancer cells) after binding to the transferrin receptor on cell surface of the cancer cells. In some embodiments, the antibody is capable of inducing apoptosis of the cancer cells after binding to the transferrin receptor on cell surface of the cancer cells in the absence of cytotoxin conjugation and immune effector function. In some embodiments, the antibody is conjugated to a cytotoxin. In some embodiments, the antibody is conjugated to a label.

Also provided herein are pharmaceutical compositions comprising any of the antibodies described herein and a pharmaceutically acceptable carrier. In some embodiments, there is provided a polynucleotide comprising a nucleic acid sequence encoding any of the antibodies described herein. In some embodiments, there is provided a vector comprising a nucleic acid sequence encoding any of the antibodies described herein. In some embodiments, there is provided a host cell comprising any of the vectors described herein. In some embodiments, there is provided a method of producing an antibody comprising culturing a host cell described herein that produces an antibody described herein and recovering the antibody produced by the host cell. In some embodiments, the antibody is isolated or purified (e.g., after being produced by the host cell).

Also provided herein are methods of treating nonhematopoietic cancer in an individual comprising administering to the individual an effective amount of an antibody described herein. Also provided herein are methods of treating nonhematopoietic cancer in an individual comprising administering to the individual an amount of an antibody described herein and an amount of another anti-cancer agent, whereby the antibody and the anti-cancer agent in conjunction provide effective treatment of cancer in the individual. In some embodiments, the anti-cancer agent is a chemotherapeutic agent. In some embodiments, the antibody is conjugated to a cytotoxin. In some embodiments, the nonhematopoietic cancer is pancreatic cancer, gastric cancer, colorectal cancer, lung cancer, ovarian cancer, prostate cancer, endometrial cancer, breast cancer, or liver cancer. In some embodiments, the individual is a human.

Also provided herein are kits comprising any of the antibodies described herein. In some embodiments, the kit further comprises instructions for administering an effective amount of the antibody to an individual for treating nonhematopoietic cancer. In some embodiments, the kit further comprises instructions for administering an amount of the antibody and an amount of another anti-cancer agent to an individual for treating nonhematopoietic cancer, whereby the antibody and the anti-cancer agent in conjunction provide effective treatment of cancer in the individual. In some embodiments, the kits further comprise a second anti-cancer agent.

Also provided herein are methods of screening an antibody that specifically binds to a transferrin receptor expressed by nonhematopoietic cancer cells comprising the steps of a) providing multiple antibodies and selecting one or more antibodies that specifically bind to a transferrin receptor expressed by nonhematopoietic cancer cells and b) using the one or more antibodies selected from step a) to further select an antibody that does not specifically bind to a transferrin receptor expressed by activated T cells or by Jurkat cells. In some embodiments, the antibody specifically binds to a carbohydrate on the transferrin receptor expressed by nonhematopoietic cancer cells. In some embodiments, the method further comprises the step of selecting the antibody that is capable of inducing apoptosis of the cancer cells after binding to transferrin receptor on cell surface of the cancer cells in the absence of cytotoxin conjugation and immune effector function. In some embodiments, the nonhematopoietic cancer cells are pancreatic cancer cells, gastric cancer cells, colorectal cancer cells, lung cancer cells, ovarian cancer cells, prostate cancer cells, endometrial cancer cells, breast cancer cells, or liver cancer cells.

It is to be understood that one, some, or all of the properties of the various embodiments described herein may be combined to form other embodiments of the present invention. These and other aspects of the invention will become apparent to one of skill in the art.

### BRIEF DESCRIPTION OF THE FIGURES

FIG. 1 is a western blot demonstrating that antibodies from 6-90, 55-31, 122-72 and 5D7-54.17 hybridoma clones were able to recognize cancer-specific human transferrin receptor. (A) immunoprecipitation and western blotting of lung cancer cells H358 and prostate cancer cells DU145. (B) immunoprecipitation and western blotting of normal activated T cells. Transferrin receptor (TfR) protein is indicated by arrow. The primary antibodies used in this experiment are shown in the figure. The secondary antibody used in this experiment was horseradish peroxidase-conjugated rabbit anti-goat IgG or goat anti-mouse IgG (H+ L), which cross-reacts with mouse IgM.

FIG. 2 depicts the amino acid sequence and nucleotide sequence for the variable region of heavy chain (A) and light chain (B) for antibody 6-90. The signal peptide of each chain is italicized and underlined. The CDRs in each chain are bolded and underlined.

FIG. 3 depicts the amino acid sequence and nucleotide sequence for the variable region of heavy chain (A) and light chain (B) for antibody 55-31. The signal peptide of each chain is italicized and underlined. The CDRs in each chain are bolded and underlined.

FIG. 4 depicts the amino acid sequence and nucleotide sequence for the variable region of heavy chain (A) and light chain (B) for antibody 122-72. The signal peptide of each chain is italicized and underlined. The CDRs in each chain are bolded and underlined.

FIG. 5 depicts the amino acid sequence and nucleotide sequence for the variable region of heavy chain (A) and light chain (B) for antibody 5D7-54.17. The signal peptide of each chain is italicized and underlined. The CDRs in each chain are bolded and underlined.

FIG. 6 shows results of internalization analysis demonstrating that antibodies from 6-90, 55-31, 122-72 and 5D7-54.17 hybridoma clones can induce internalization in Panc 02.03B, H358, DLD-1 and OMC-3 cancer cell lines.

FIG. 7 is a western blot identifying the sialyl moiety recognized by 122-72 and 5D7-54.17 antibodies as demonstrated by loss of antibody recognition to α2-3,6,8-Neuraminidase treated rCEA protein. The primary antibodies used in this experiment are shown in the figure. The secondary antibody used here is anti-mouse IgG (H+L)-HRP, which cross-reacts with mouse IgM.

FIG. 8 is a western blot identifying the fucose moiety recognized by 6-90 antibody as demonstrated by loss of antibody recognition to α-1→(2,3,4)-fucosidase and N-glycanase treated rCEA protein. The primary antibodies used in this experiment are shown in the figure. The secondary antibody used here is anti-mouse IgG (H+L)-HRP, which cross-reacts with mouse IgM.

FIG. 9 is a graph depicting the percent inhibition of binding for 6-90, 55-31, 122-72 and 5D7-54.17 antibodies to pancreatic cancer Panc 02.03B cells by competition with Lewis^{a} glycan.

FIG. 10 depicts in vivo anti-tumor activity of c5D7-conjugated ADC against colorectal cancer DLD-1.

### DETAILED DESCRIPTION

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibody of the present invention is further intended to include bispecific, multispecific, single-chain, and chimeric and humanized molecules having affinity for a polypeptide conferred by at least one hypervariable region (HVR) or complementarity determining region (CDR) of the antibody. Antibodies of the present invention also include single domain antibodies which are either the variable domain of an antibody heavy chain or the variable domain of an antibody light chain. Holt et al., Trends Biotechnol. 21:484-490, 2003. Methods of making domain antibodies comprising either the variable domain of an antibody heavy chain or the variable domain of an antibody light chain, containing three of the six naturally occurring HVRs or CDRs from an antibody, are also known in the art. See, e.g., Muyldermans, Rev. Mol. Biotechnol. 74:277-302, 2001. In some embodiments, the antibody of the present invention encompasses an antibody that is conjugated to an agent such as cytotoxin.

As used herein, “monoclonal antibody” refers to an antibody of substantially homogeneous antibodies, i.e., the individual antibodies comprising the population are identical except for possible naturally-occurring mutations that may be present in minor amounts. Furthermore, in contrast to polyclonal antibody preparations, which typically include different antibodies directed against different determinants (epitopes), monoclonal antibody is not a mixture of discrete antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, a “chimeric antibody” refers to an antibody having a variable region or part of variable region from a first species and a constant region from a second species. An intact chimeric antibody comprises two copies of a chimeric light chain and two copies of a chimeric heavy chain. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human is less likely to elicit an immune response from a human subject when the antibodies are injected than would the constant region from a non-human source. However, the definition is not limited to this particular example.

An “isolated” antibody is one which has been identified and separated and/or recovered from a component of its natural environment.

As used herein, “substantially pure” refers to material which is at least 50% pure (i.e., free from contaminants), more preferably at least 90% pure, more preferably at least 95% pure, more preferably at least 98% pure, more preferably at least 99% pure.

As used herein, “humanized” antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a HVR or CDR of the recipient are replaced by residues from a HVR or CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported HVR or CDR or framework sequences, but are included to further refine and optimize antibody performance. In general, the humanized antibody will comprise substantially all of at least one, and typically two, variable domains, in which all or substantially all of the HVR or CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more HVRs or CDRs (one, two, three, four, five, six) which are altered with respect to the original antibody, which are also termed one or more HVRs or CDRs “derived from” one or more HVRs or CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. Generally, the variable region(s) mediate antigen binding and define specificity of a particular antibody for its particular antigen. The variable regions may have relatively invariant stretches called framework regions (FRs) (e.g., FR of 15-30 amino acids) separated by shorter regions of extreme variability called “hypervariable regions” (“HVR”) (e.g., HVRs that are each 9-12 amino acids long). In some embodiments, the variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain may be held together in close proximity by the FRs and, with the hypervariable regions from the other chain, contribute to the formation of the antigen-binding site of antibodies (see Kabat et al., Sequences of Proteins of Immunological Interest. 5th Ed. Public Health Service, National Institutes of Health, Bethesda, Md. (1991)). The constant domains may not be involved directly in binding an antibody to an antigen, but may exhibit various effector functions, such as participation of the antibody in antibody dependent cellular cytotoxicity (ADCC).

The term “hypervariable region” (“HVR”) when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the VL, and around about 31-35B (H1), 50-65 (H2) and 95-102 (H3) in the VJJ (in one embodiment, H1 is around about 31-35); Kabat et al., Sequences of Proteins of Immunological Interest. 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 53-55 (H2) and 96-101 (H3) in the VH; Chothia and Lesk J. Mol. Biol. 196:901-917 (1987)). There are multiple ways for determining CDRs, for example, an approach based on cross-species sequence variability (i.e., Kabat et al. Sequences of Proteins of Immunological Interest, (5th ed., 1991, National Institutes of Health, Bethesda Md.)); and an approach based on crystallographic studies of antigen-antibody complexes (Al-lazikani et al. (1997) J. Molec. Biol. 273:927-948)). The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. As used herein, a CDR may be CDRs defined by any of the approaches or by a combination of any two or three of the approaches. The CDR may be Kabat CDR, Chothia CDR, or contact CDR. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

A “constant region” of an antibody refers to the constant region of the antibody light chain or the constant region of the antibody heavy chain, either alone or in combination. A constant region of an antibody generally provides structural stability and other biological functions such as antibody chain association, secretion, transplacental mobility, and complement binding, but is not involved with binding to the antigen. The amino acid sequence and corresponding exon sequences in the genes of the constant region will be dependent upon the species from which it is derived; however, variations in the amino acid sequence leading to allotypes will be relatively limited for particular constant regions within a species. The variable region of each chain is joined to the constant region by a linking polypeptide sequence. The linkage sequence is coded by a “J” sequence in the light chain gene, and a combination of a “D” sequence and a “J” sequence in the heavy chain gene.

As used herein, “antibody-dependent cell-mediated cytotoxicity” and “ADCC” refer to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods, 202:163 (1996), may be performed.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. In some embodiments, the term “effective dosage” or “effective amount” refers to an amount of an antibody or polypeptide described herein that is sufficient to effect beneficial or desired results.

As used herein, “in conjunction with” refers to administration of one treatment modality in addition to another treatment modality. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of this invention, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “individual” or a “subject” is a mammal, more preferably a human. Mammals also include, but are not limited to, farm animals, sport animals, pets (such as cats, dogs, horses), primates, mice and rats.

As use herein, the term “specifically recognizes” or “specifically binds” refers to measurable and reproducible interactions such as attraction or binding between a target and an antibody, that is determinative of the presence of the target in the presence of a heterogeneous population of molecules including biological molecules. For example, an antibody that specifically or preferentially binds to an epitope is an antibody that binds this epitope with greater affinity, avidity, more readily, and/or with greater duration than it binds to other epitopes of the target or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “cancer,” “tumor,” “cancerous,” and “malignant” refer to or describe the physiological condition in mammals that is typically characterized by unregulated cell growth. Examples of cancer include but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, prostate cancer, thyroid cancer, testicular cancer, esophageal cancer, and various types of head and neck cancer.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

Antibodies that Specifically Bind to Transferrin Receptor Expressed by Nonhematopoietic Cancer Cells

Provided herein are antibodies, and polypeptides derived from the antibodies, that specifically bind to a transferrin receptor (such as human transferrin receptor) expressed by nonhematopoietic cancer cells. The antibodies or polypeptides may specifically bind to a modification (such as a carbohydrate) on a transferrin receptor expressed by nonhematopoietic cancer cells. In some embodiments, the transferrin receptor is a human transferrin receptor, e.g., a human transferrin receptor with amino acid sequence set forth in GenBank Accession No. AAA61153.1 or GenBank Accession No. AAF04564.1 (the contents of which are incorporated herein by references in their entirety), or a human transferrin receptor with amino acid sequence set forth in SEQ ID NO:19 (see below). In some embodiments, the antibody or polypeptide specifically binds to a modification (such as a carbohydrate) on a transferrin receptor with amino acids 1-760 in SEQ ID NO:19 or a portion thereof.

| (SEQ ID NO: 19) | |
|---|---|
| 1 | mmdqarsafs nlfggeplsy trfslarqvd gdnshvemkl avdeeenadn ntkanvtkpk |
| 61 | rcsgsicygt iavivfflig fmigylgyck gvepktecer lagtespvre epgedfpaar |
| 121 | rlywddlkrk lsekldstdf tstikllnen syvpreagsq kdenlalyve nqfrefklsk |
| 181 | vwrdqhfvki qvkdsaqnsv iivdkngrlv ylvenpggyv ayskaatvtg klvhanfgtk |
| 241 | kdfedlytpv ngsivivrag kitfaekvan aeslnaigvl iymdqtkfpi vnaelsffgh |
| 301 | ahlgtgdpyt pgfpsfnhtq fppsrssglp nipvqtisra aaeklfgnme gdcpsdwktd |
| 361 | stcrmvtses knvkltvsnv lkeikilnif gvikgfvepd hyvvvgaqrd awgpgaaksg |
| 421 | vgtalllkla qmfsdmvlkd gfqpsrsiif aswsagdfgs vgatewlegy lsslhlkaft |
| 481 | yinldkavlg tsnfkvsasp llytliektm qnvkhpvtgq flyqdsnwas kvekltldna |
| 541 | afpflaysgi paysfcfced tdypylgttm dtykelieri pelnkvaraa aevagqfvik |
| 601 | lthdvelnld yerynsqlls fvrdlnqyra dikemglslq wlysargdff ratsrlttdf |
| 661 | gnaektdrfv mkklndrvmr veyhflspyv spkespfrhv fwgsgshtlp allenlklrk |
| 721 | qnngafnetl frnqlalatw tiqgaanals gdvwdidnef |

Provided herein are antibodies or polypeptides that specifically bind to a carbohydrate on a transferrin receptor expressed by nonhematopoietic cancer cells (such as pancreatic cancer cells, gastric cancer cells, colorectal cancer cells, lung cancer cells, ovarian cancer cells, endometrial cancer cells, gallbladder cancer cells, prostate cancer cells, breast cancer cells, or liver cancer cells). The antibodies or polypeptides may not specifically bind to a transferrin receptor expressed by activated T cells (such as peripheral activated T cells) or Jurkat cells. In some embodiments, the binding of the antibody to the transferrin receptor expressed by nonhematopoietic cancer cells is not inhibited by a carbohydrate comprising a Lewis a (Le^{a}) structure. In some embodiments, the cancer cells are human cancer cells. In some embodiments, the transferrin receptor is a mammalian transferrin receptor (such as a human transferrin receptor or a mouse transferrin receptor). In some embodiments, the transferrin receptor is a human transferrin receptor. In some embodiments, a modification on a transferrin receptor described herein refers to a modification (e.g., a carbohydrate) on the transferrin receptor expressed by the cancer cells, wherein said modification is not present on a transferrin receptor expressed by activated T cells or Jurkat cells.

In some embodiments, the antibody or the polypeptide binds to an epitope comprising a fucose moiety. In some embodiments, the antibody or the polypeptide binds to an epitope that does not comprise a fucose moiety. In some embodiments, the antibody or the polypeptide binds to an epitope comprising a sialyl moiety. In some embodiments, the antibody or the polypeptide binds to an epitope that does not comprise a sialyl moiety.

The antibodies and polypeptides provided herein may further have one or more of the following characteristics: (i) binding of the antibody or the polypeptide to the epitope on the transferrin receptor is reduced if the molecule comprising the epitope is treated with α2-3,6,8-Neuraminidase, α-1→(2,3,4)-Fucosidase, or N-glycanase; (ii) binding of the antibody or the polypeptide to the epitope is not inhibited by a carbohydrate comprising a Lewis a (Le^{a}) structure (such as trisaccharide lewis a); (iii) binding of the antibody or the polypeptide to the epitope is not inhibited by a carbohydrate comprising a Lewis b (Le^{b}), Lewis y (Le^{y}), or Lewis x (Le^{x})) structure; (iv) induce death of the nonhematopoietic cancer cell (such as through apoptosis) after binding to the epitope expressed on the cell surface of the cancer cell (e.g., in vitro) in the absence of cytotoxin conjugation and immune effector function; and (v) the antibody or the polypeptide does not bind to a transferrin receptor expressed by CHO cells, red blood cells, platelets, HUVEC cells, monocytes, PMN, lymphocytes, Jurkat cells, T cells, activated T cells, B cells, leukemia cells, T leukemia cells, or B leukemia cells. As used herein, the term “inhibition” includes partial and complete inhibition. Binding of the antibody to the epitope may be inhibited by direct competition or by other mechanisms. The antibodies and polypeptides provided herein may inhibit cell growth or proliferation of the cancer cell (such as nonhematopoietic cancer cell) after binding to the epitope expressed on the cell surface of the cancer cell; and/or treat or prevent cancer cell (such as nonhematopoietic cancer) expressing the epitope on the cell surface. The cancer cells may be human cancer cells such as human nonhematopoietic cancer cells. The nonhematopoietic cancer cells may be any of pancreatic cancer cells, gastric cancer cells, colorectal cancer cells, lung cancer cells, ovarian cancer cells, endometrial cancer cells, prostate cancer cells, breast cancer cells, or liver cancer cells. Examples of non-hematopoietic cancer cells expressing the epitope include, but are not limited to, lung cancer cells (such as H358, A549, H520, and H727), pancreatic cancer cells (such as Panc 02.03B, SU.86.86, and Panc-1), gastric cancer cells (such as SNU-16, NCI-N87, and Kato III), ovarian cancer cells (such as OMC-3 and SK-OV-3), endometrial cancer cells (such as HEC-1-A and KLE), colorectal cancer cells (such as COLO 205, WiDr, and DLD-1), breast cancer cells (such as MDA-MB-453, Hs578T, and T47D), prostate cancer cells (DU145, PC3, and 22Rv1), and liver cancer cells (such as PLC/PRF/5, Hep G2, and Hep 3B2.1-7).

The antibodies and polypeptides of the present invention may recognize an extracellular domain of a transferrin receptor expressed or present on a nonhematopoietic cancer cell, but does not bind to an extracellular domain of a transferrin receptor expressed or present on leukocyte (e.g., a peripheral T cell) or on a Jurkat cell (a lymphoblastoid leukemia cell). In some embodiments, the antibodies or polypeptides provided herein do not specifically bind to a transferrin receptor expressed by a cell of hematopoietic origin.

The antibodies of the invention can encompass monoclonal antibodies, polyclonal antibodies, antibody fragments (e.g., Fab, Fab′, F(ab′)₂, Fv, Fc, etc.), chimeric antibodies, single chain (ScFv), bispecific antibodies, antibodies that are conjugated to cytotoxin, mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies).

The binding affinity of the polypeptide (including antibody) to a transferrin receptor expressed on a nonhematopoietic cancer cell may be less than any of about 500 nM, about 400 nM, about 300 nM, about 200 nM, about 100 nM, about 50 nM, about 10 nM, about 1 nM, about 500 pM, about 100 pM, or about 50 pM. As is well known in the art, binding affinity can be expressed as K_{D}, or dissociation constant, and an increased binding affinity corresponds to a decreased K_{D}. One way of determining binding affinity of antibodies to transferrin receptor is by measuring binding affinity of monofunctional Fab fragments of the antibody. To obtain monofunctional Fab fragments, an antibody (for example, IgG) can be cleaved with papain or expressed recombinantly. The affinity of an Fab fragment of an antibody can be determined by surface plasmon resonance (BIAcore3000™ surface plasmon resonance (SPR) system, BIAcore, INC, Piscaway, N.J.) and ELISA. Kinetic association rates (kₒₙ) and dissociation rates (k_{off}) (generally measured at 25° C.) are obtained; and equilibrium dissociation constant (K_{D}) values are calculated as k_{off}/kₒₙ.

In some embodiments, the antibodies and polypeptides provided herein reduce the number of cancer cells, and/or inhibit cell growth or proliferation of tumor or cancer cells expressing the transferrin receptor to which the antibodies and polypeptides recognize. Preferably, the reduction in cell number or inhibition of cell growth or proliferation is by at least about 10%, about 20%, about 30%, about 40%, about 50%, about 65%, about 75%, or greater as compared to the cell not treated with the antibody or polypeptides. Cancer cells include, but are not limited to, pancreatic cancer, gastric cancer, colorectal cancer cells, lung cancer, ovarian cancer, endometrial cancer, prostate cancer, breast cancer, gallbladder cancer, or liver cancer.

In some embodiments, the antibodies and polypeptides provided herein are capable of inducing cell death alone, for example through apoptosis, after binding to transferrin receptor expressed by nonhematopoietic cancer cells. The term “induce cell death” as used herein, means that the antibodies or polypeptides of the present disclosure, can directly interact with a molecule expressed on the cell surface, and the binding/interaction alone is sufficient to induce cell death in the cells without the help of other factors such as cytotoxin conjugation or other immune effector functions, i.e., complement-dependent cytotoxicity (CDC), antibody-dependent cellular cytotoxicity (ADCC), or phagocytosis. In some embodiments, the antibodies and polypeptides provided herein are capable of being internalized (e.g., internalized in cancer cells) after binding to transferrin receptor on cell surface of the cancer cells.

As used herein, the term “apoptosis” refers to gene-directed process of intracellular cell destruction. Apoptosis is distinct from necrosis; it includes cytoskeletal disruption, cytoplasmic shrinkage and condensation, expression of phosphatidylserine on the outer surface of the cell membrane and blebbing, resulting in the formation of cell membrane bound vesicles or apoptotic bodies. The process is also referred to as “programmed cell death.” During apoptosis, characteristic phenomena such as curved cell surfaces, condensation of nuclear chromatin, fragmentation of chromosomal DNA, and loss of mitochondrial function are observed. Various known technologies may be used to detect apoptosis, such as staining cells with Annexin V, propidium iodide, DNA fragmentation assay and YO-PRO-1 (Invitrogen).

Methods of detecting cell death (such as apoptosis) include, but are not limited to, detecting morphology, DNA fragmentation, enzymatic activity, and polypeptide degradation, etc. See Siman et al., U.S. Pat. No. 6,048,703; Martin and Green (1995), Cell, 82: 349-52; Thomberry and Lazebnik (1998), Science, 281:1312-6; Zou et al., U.S. Pat. No. 6,291,643; Scovassi and Poirier (1999), Mol. Cell. Biochem., 199: 125-37; Wyllie e tal. (1980), Int. Rev. Cytol., 68:251-306; Belhocine et al. (2004), Technol. Cancer Res. Treat., 3(1):23-32, which are incorporated herein by reference.

In some embodiments, the antibodies and polypeptides provided herein competes with antibody 6-90, 55-31, 122-72, 5D7-54.17, or chimeric antibody derived from 5D7-54.17 (c5D7), for binding to the transferrin receptor expressed on the cell surface of the cancer cell. In some embodiments, the antibodies or polypeptides of the invention binding to an epitope on transferrin receptor to which at least one of antibodies 6-90, 55-31, 122-72, 5D7-54.17 and c5D7 binds.

In some embodiments, competition assays may be used to identify a monoclonal antibody that competes with an antibody or polypeptide described herein (such as 6-90, 55-31, 122-72, 5D7-54.17, and c5D7) for binding to transferrin receptor expressed by cancer cells. The cancer cells may be any of the cancer cells described herein. Competition assays can be used to determine whether two antibodies bind the same epitope by recognizing identical or sterically overlapping epitopes or one antibody competitively inhibits binding of another antibody to the antigen. In certain embodiments, such a competing antibody binds to the same epitope (e.g., a carbohydrate on transferrin receptor as described herein) that is bound by an antibody or polypeptide described herein (such as 6-90, 55-31, 122-72, 5D7-54.17, and c5D7). Exemplary competition assays include, but are not limited to, routine assays such as those provided in Harlow and Lane (1988) Antibodies: A Laboratory Manual ch.14 (Cold Spring Harbor Laboratory, Cold Spring Harbor, N.Y.). Detailed exemplary methods for mapping an epitope to which an antibody binds are provided in Morris (1996) “Epitope Mapping Protocols,” in Methods in Molecular Biology vol. 66 (Humana Press, Totowa, N.J.). Two antibodies are said to bind to the same epitope if each blocks binding of the other by 50% or more.

In an exemplary competition assay, immobilized transferrin receptor (TfR) expressed by cancer cells is incubated in a solution comprising a first labeled antibody that binds to the TfR (such as 6-90, 55-31, 122-72, 5D7-54.17, and c5D7) and a second unlabeled antibody that is being tested for its ability to compete with the first antibody for binding to the TfR. The second antibody may be present in a hybridoma supernatant. As a control, immobilized TfR is incubated in a solution comprising the first labeled antibody but not the second unlabeled antibody. After incubation under conditions permissive for binding of the first antibody to TfR, excess unbound antibody is removed, and the amount of label associated with immobilized TfR is measured. If the amount of label associated with immobilized TfR is substantially reduced in the test sample relative to the control sample, then that indicates that the second antibody is competing with the first antibody for binding to TfR. In certain embodiments, immobilized TfR is present on the surface of a cell or in a membrane preparation obtained from a cell expressing TfR on its surface. Common labels for such competition assays may be radioactive labels or enzyme labels.

In some embodiments, the antibody of the invention is antibody 6-90 or an antibody derived from 6-90. The heavy chain and light chain variable sequences of 6-90 are set forth in SEQ ID NO:1 and SEQ ID NO:3, respectively. In some embodiments, the antibody of the invention competes or specifically competes for binding to transferrin receptor expressed by nonhematopoietic cancer cells with an antibody comprising one, two, or three HVRs (or CDRs) from a light chain and/or a heavy chain of the antibody 6-90 (or an antibody derived from 6-90). The invention further provides an antibody or a polypeptide comprising a fragment or a region of the antibody 6-90. In one embodiment, the fragment is a light chain of the antibody 6-90. In another embodiment, the fragment is a heavy chain of the antibody 6-90. In yet another embodiment, the fragment contains one or more variable regions from a light chain and/or a heavy chain of the antibody 6-90 (or an antibody derived from 6-90). In yet another embodiment, the fragment contains one, two, or three HVRs (or CDRs) from a light chain and/or a heavy chain of the antibody 6-90 (or an antibody derived from 6-90). In some embodiments, the one or more HVRs (or CDRs) derived from antibody 6-90 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of 6-90. In some embodiments, the antibody comprises a heavy chain variable region comprising the three HVRs (or CDRs) from (or of) SEQ ID NO:1 and/or a light chain variable region comprising the three HVRs (or CDRs) from (or of) SEQ ID NO:3.

In some embodiments, the antibody comprises (i) a heavy chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 20-138 of SEQ ID NO:1, and/or (ii) a light chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 20-132 of SEQ ID NO:3. In some embodiments, the antibody comprises a heavy chain variable region comprising amino acids 20-138 of SEQ ID NO:1 and/or a light chain variable region comprising amino acids 20-132 of SEQ ID NO:3.

In some embodiments, the antibody of the invention is antibody 55-31 or an antibody derived from 55-31. The heavy chain and light chain variable sequences of 55-31 are set forth in SEQ ID NO:5 and SEQ ID NO:7, respectively. In some embodiments, the antibody of the invention competes or specifically competes for binding to transferrin receptor expressed by nonhematopoietic cancer cells with an antibody comprising one, two, or three HVRs (or CDRs) from a light chain and/or a heavy chain of the antibody 55-31 (or an antibody derived from 55-31). The invention further provides an antibody or a polypeptide comprising a fragment or a region of the antibody 55-31. In one embodiment, the fragment is a light chain of the antibody 55-31. In another embodiment, the fragment is a heavy chain of the antibody 55-31. In yet another embodiment, the fragment contains one or more variable regions from a light chain and/or a heavy chain of the antibody 55-31 (or an antibody derived from 55-31). In yet another embodiment, the fragment contains one, two, or three HVRs (or CDRs) from a light chain and/or a heavy chain of the antibody 55-31 (or an antibody derived from 55-31). In some embodiments, the one or more HVRs (or CDRs) derived from antibody 55-31 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of 55-31. In some embodiments, the antibody comprises a heavy chain variable region comprising the three HVRs (or CDRs) from (or of) SEQ ID NO:5 and/or a light chain variable region comprising the three HVRs (or CDRs) from (or of) SEQ ID NO:7.

In some embodiments, the antibody comprises (i) a heavy chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 20-138 of SEQ ID NO:5, and/or (ii) a light chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 21-128 of SEQ ID NO:7. In some embodiments, the antibody comprises a heavy chain variable region comprising amino acids 20-138 of SEQ ID NO:5 and/or a light chain variable region comprising amino acids 21-128 of SEQ ID NO:7.

In some embodiments, the antibody of the invention is antibody 122-72 or an antibody derived from 122-72. The heavy chain and light chain variable sequences of 122-72 are set forth in SEQ ID NO:9 and SEQ ID NO:11, respectively. In some embodiments, the antibody of the invention competes or specifically competes for binding to transferrin receptor expressed by nonhematopoietic cancer cells with an antibody comprising one, two, or three HVRs (or CDRs) from a light chain and/or a heavy chain of the antibody 122-72 (or an antibody derived from 122-72). The invention further provides an antibody or a polypeptide comprising a fragment or a region of the antibody 122-72. In one embodiment, the fragment is a light chain of the antibody 122-72. In another embodiment, the fragment is a heavy chain of the antibody 122-72. In yet another embodiment, the fragment contains one or more variable regions from a light chain and/or a heavy chain of the antibody 122-72 (or an antibody derived from 122-72). In yet another embodiment, the fragment contains one, two, or three HVRs (or CDRs) from a light chain and/or a heavy chain of the antibody 122-72 (or an antibody derived from 122-72). In some embodiments, the one or more HVRs (or CDRs) derived from antibody 122-72 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of 122-72. In some embodiments, the antibody comprises a heavy chain variable region comprising the three HVRs (or CDRs) from (or of) SEQ ID NO:9 and/or a light chain variable region comprising the three HVRs (or CDRs) from (or of) SEQ ID NO:11.

In some embodiments, the antibody comprises (i) a heavy chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 20-136 of SEQ ID NO:9, and/or (ii) a light chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 21-134 of SEQ ID NO:11. In some embodiments, the antibody comprises a heavy chain variable region comprising amino acids 20-136 of SEQ ID NO:9 and/or a light chain variable region comprising amino acids 21-134 of SEQ ID NO:11.

In some embodiments, the antibody of the invention is antibody 5D7-54.17 or an antibody derived from 5D7-54.17. The heavy chain and light chain variable sequences of 5D7-54.17 are set forth in SEQ ID NO:13 and SEQ ID NO:15, respectively. In some embodiments, the antibody of the invention competes or specifically competes for binding to transferrin receptor expressed by nonhematopoietic cancer cells with an antibody comprising one, two, or three HVRs (or CDRs) from a light chain and/or a heavy chain of the antibody 5D7-54.17 (or an antibody derived from 5D7-54.17). The invention further provides an antibody or a polypeptide comprising a fragment or a region of the antibody 5D7-54.17. In one embodiment, the fragment is a light chain of the antibody 5D7-54.17. In another embodiment, the fragment is a heavy chain of the antibody 5D7-54.17. In yet another embodiment, the fragment contains one or more variable regions from a light chain and/or a heavy chain of the antibody 5D7-54.17 (or an antibody derived from 5D7-54.17). In yet another embodiment, the fragment contains one, two, or three HVRs (or CDRs) from a light chain and/or a heavy chain of the antibody 5D7-54.17 (or an antibody derived from 5D7-54.17). In some embodiments, the one or more HVRs (or CDRs) derived from antibody 5D7-54.17 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of 5D7-54.17. In some embodiments, the antibody comprises a heavy chain variable region comprising the three HVRs (or CDRs) from (or of) SEQ ID NO:13 and/or a light chain variable region comprising the three HVRs (or CDRs) from (or of) SEQ ID NO:15.

In some embodiments, the antibody comprises (i) a heavy chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to 20-138 of SEQ ID NO:13, and/or (ii) a light chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 23-130 of SEQ ID NO:15. In some embodiments, the antibody comprises a heavy chain variable region comprising amino acids 20-138 of SEQ ID NO:13 and/or a light chain variable region comprising amino acids 23-130 of SEQ ID NO:15.

In some embodiments, the antibody of the invention is a chimeric antibody derived from antibody 5D7-54.17 (c5D7) or an antibody derived from c5D7 antibody. The heavy chain and light chain sequences of c5D7 antibody are set forth in SEQ ID NO:17 and SEQ ID NO:18, respectively. In some embodiments, the antibody of the invention competes or specifically competes for binding to transferrin receptor expressed by nonhematopoietic cancer cells with an antibody comprising one, two, or three HVRs (or CDRs) from a light chain and/or a heavy chain of the antibody c5D7 (or an antibody derived from c5D7). The invention further provides an antibody or a polypeptide comprising a fragment or a region of the antibody c5D7 (or an antibody derived from c5D7). In one embodiment, the fragment is a light chain of the antibody c5D7 (or an antibody derived from c5D7). In another embodiment, the fragment is a heavy chain of the antibody c5D7 (or an antibody derived from c5D7). In yet another embodiment, the fragment comprises one or more variable regions from a light chain and/or a heavy chain of the antibody c5D7 (or an antibody derived from c5D7). In yet another embodiment, the fragment comprises one, two, or three HVRs (or CDRs) from a light chain and/or a heavy chain of the antibody c5D7 (or an antibody derived from c5D7). In some embodiments, the one or more HVRs (or CDRs) derived from antibody c5D7 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of c5D7. In some embodiments, the antibody comprises a heavy chain variable region comprising one, two or three HVRs (or CDRs) from (or of) SEQ ID NO:17 and/or a light chain variable region comprising one, two or three HVRs (or CDRs) from (or of) SEQ ID NO:18. In some embodiments, the antibody comprises a heavy chain variable region comprising the three HVRs (or CDRs) from (or of) SEQ ID NO:17 and/or a light chain variable region comprising the three HVRs (or CDRs) from (or of) SEQ ID NO:18. In some embodiments, the antibody comprises a heavy chain variable region comprising amino acids 1-119 of SEQ ID NO:17 and/or a light chain variable region comprising amino acids 1-108 of SEQ ID NO:18. In some embodiments, the antibody is chimeric antibody. In some embodiments, the antibody is humanized antibody.

In some embodiments, the antibody comprises (i) a heavy chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 1-119 of SEQ ID NO:17, and/or (ii) a light chain variable region comprising a sequence that is at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to amino acids 1-108 of SEQ ID NO:18. In some embodiments, the antibody comprises a heavy chain variable region comprising amino acids 1-119 of SEQ ID NO:17 and/or a light chain variable region comprising amino acids 1-108 of SEQ ID NO:18.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

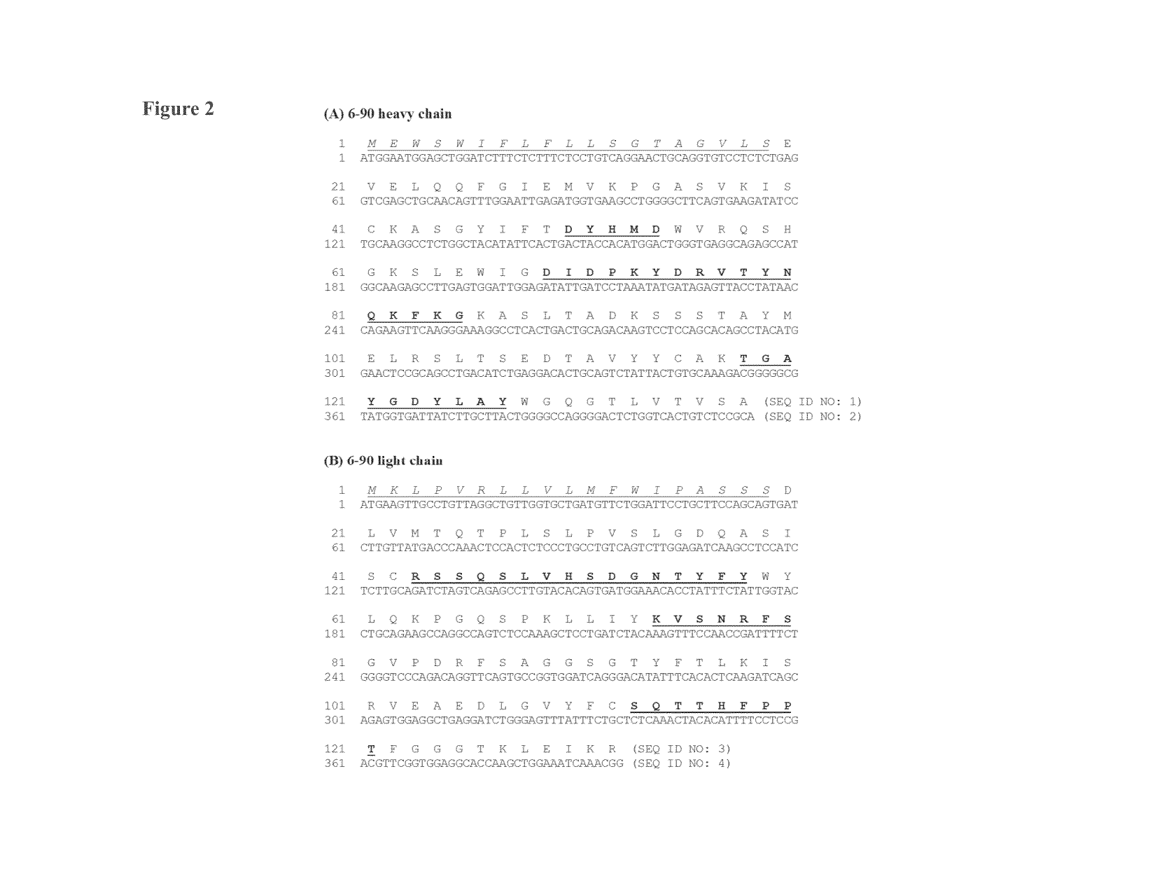

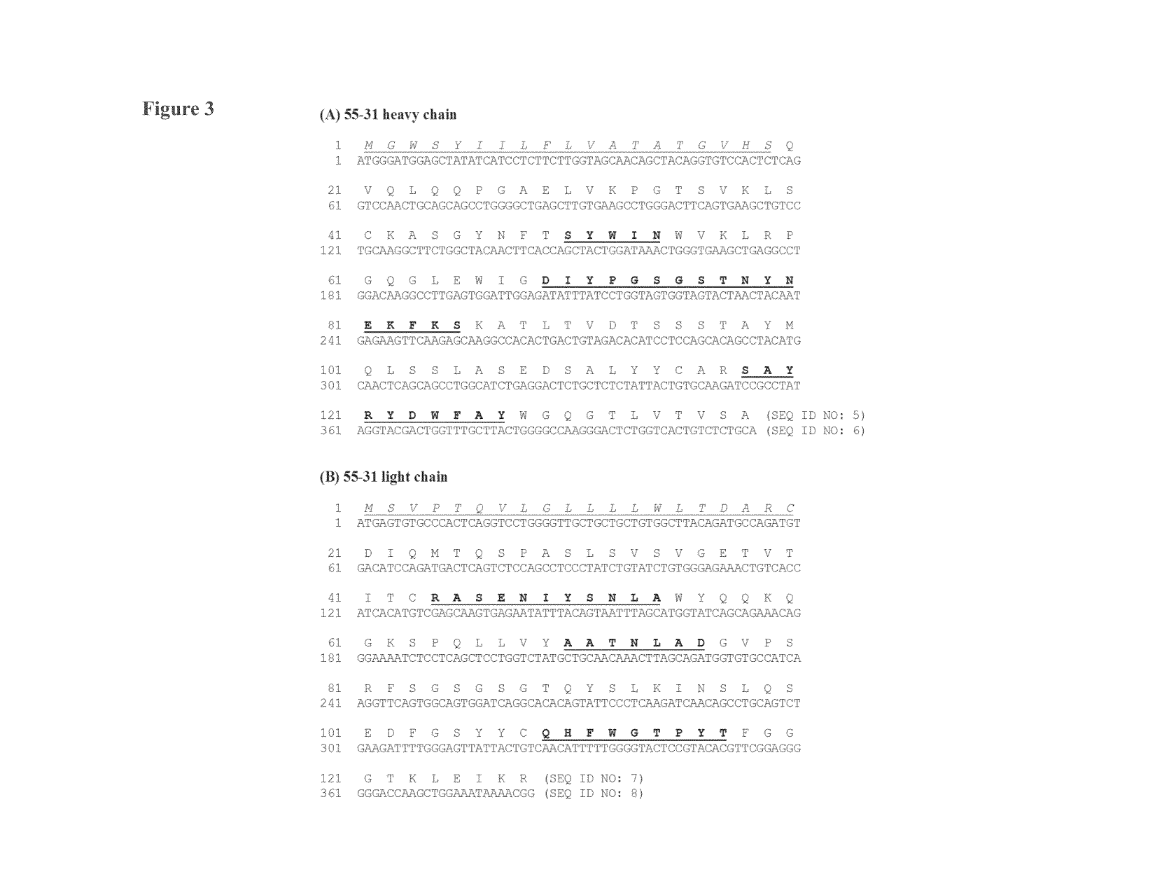

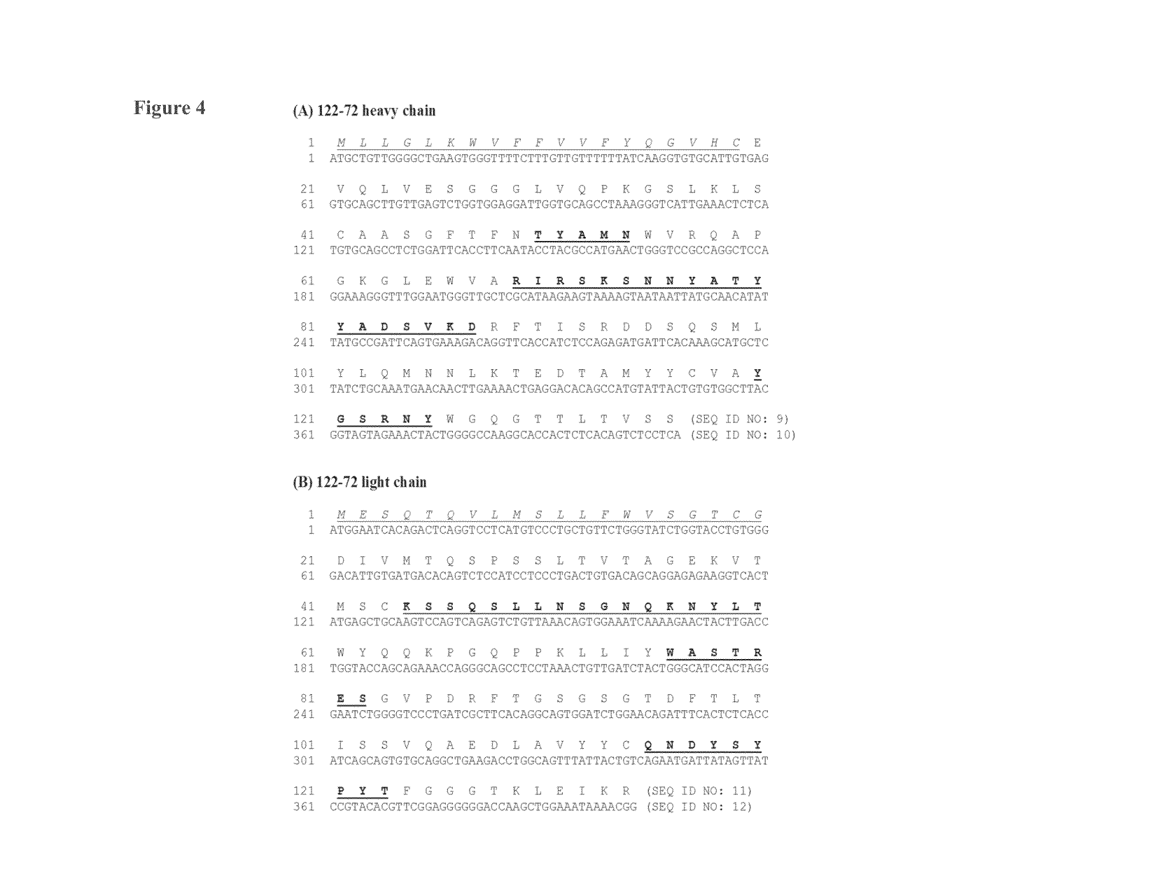

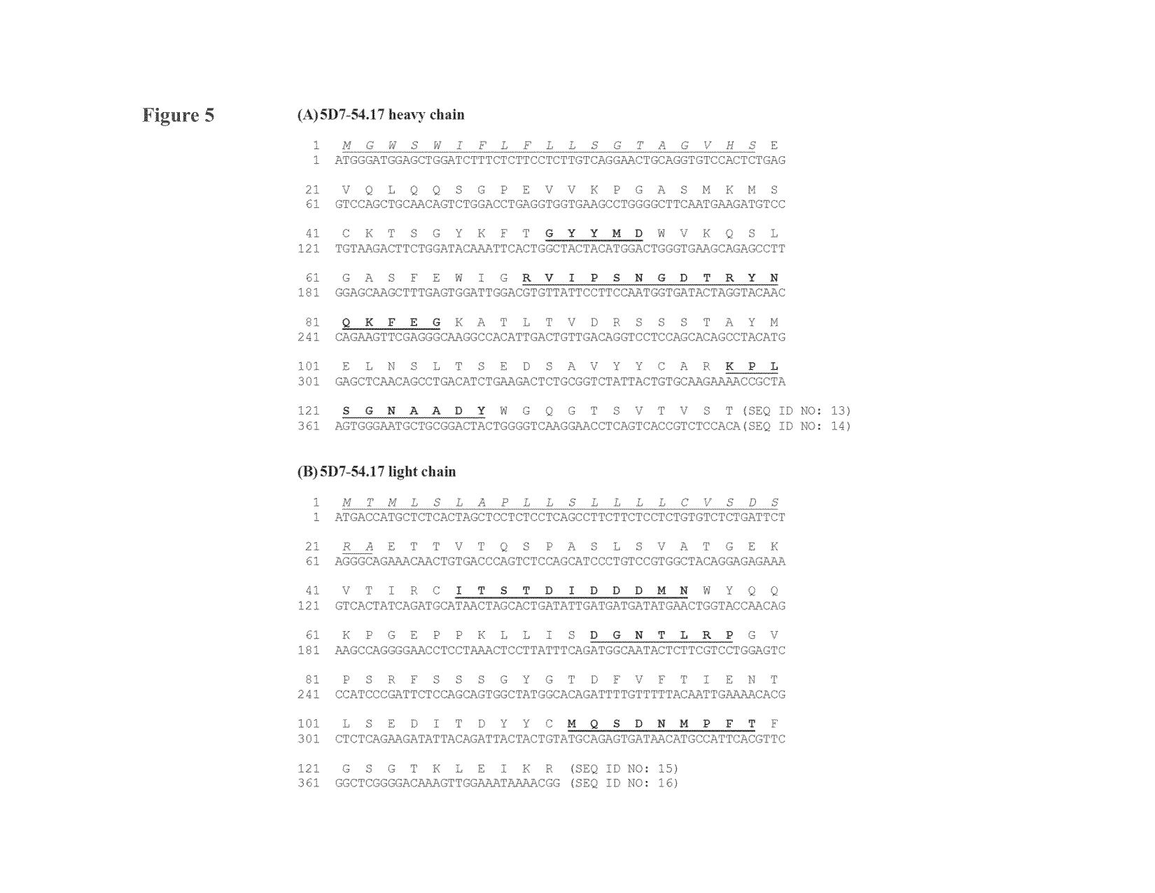

In some embodiments, a CDR described herein is Kabat CDR, Chothia CDR, or contact CDR. The Kabat CDRs for some of the antibodies provided in the present disclosure are shown in FIGS. 2-5 and Table 6. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined.

In some embodiments, the antibody or polypeptide provided herein is isolated. In some embodiments, the antibody provided herein is a monoclonal antibody. In some embodiments, the antibody is a humanized antibody. In some embodiments, the antibody is a chimeric antibody. In some embodiments, the antibody is a human antibody. In some embodiments, the antibody is IgG (such as IgG₁, IgG₂, or IgG₄). In some embodiments, the antibody is human IgG such as human IgG₁.

Also provided herein are methods of screening an antibody against cancer specific modification(s) on transferrin receptor. The method may comprise testing or screening an antibody for binding to cancer specific modification(s) on transferrin receptor, such as testing or screening an antibody for binding to cancer cells (or cancer cell fragments) or transferrin receptor isolated or purified from cancer cells. In some embodiments, the method comprises measuring the binding of an antibody to cancer specific modification(s) on transferrin receptor, such as measuring the binding of the antibody to cancer cells (or cancer cell fragments) or transferrin receptor isolated or purified from cancer cells. The method may also comprise testing or screening an antibody for no specific binding or minimal binding to cells without cancer specific modification(s) on transferrin receptor, such as normal cells (such as T cells or activated T cells), non-cancer cells, or hematopoietic cancer cells, or cell fragments thereof or transferrin receptor isolated or purified from such cells. An antibody that binds specifically to cancer specific modification(s) on transferrin receptor may show specific binding to cancer cells (or cancer cell fragments) or transferrin receptor isolated or purified from cancer cells and show no specific binding or minimal binding to cells without cancer specific modification(s) on transferrin receptor, such as normal cells (such as T cells or activated T cells), non-cancer cells, or hematopoietic cancer cells, or cell fragments thereof or transferrin receptor isolated or purified from such cells. The cancer cells may be any of the cancer cells described herein. In some embodiments, the antibody specifically binds to transferrin receptor on cancer cells that are nonhematopoietic cancer cells. In some embodiments, the antibody shows no specific binding or minimal binding to transferrin receptor on cells that are any of the following: CHO cells, red blood cells, platelets, HUVEC cells, monocytes, PMN, lymphocytes, Jurkat cells, T cells (e.g., activated T cells), B cells, leukemia cells, T leukemia cells, or B leukemia cells.

In some embodiments, there is provided a method of screening an antibody that specifically binds to a transferrin receptor expressed by nonhematopoietic cancer cells comprising the steps of a) providing multiple antibodies and selecting one or more antibodies that specifically bind to a transferrin receptor expressed by nonhematopoietic cancer cells and b) using the one or more antibodies selected from step a) to further select an antibody that does not specifically bind to a transferrin receptor expressed by activated T cells or by Jurkat cells. In some embodiments, the antibody specifically binds to a carbohydrate on the transferrin receptor expressed by nonhematopoietic cancer cells. In some embodiments, the method further comprises the step of selecting the antibody that is capable of inducing apoptosis of the cancer cells after binding to transferrin receptor on cell surface of the cancer cells in the absence of cytotoxin conjugation and immune effector function. In some embodiments, the nonhematopoietic cancer cells are pancreatic cancer cells, gastric cancer cells, colorectal cancer cells, lung cancer cells, ovarian cancer cells, endometrial cancer, prostate cancer cells, breast cancer cells, or liver cancer cells.

Methods of making antibodies and polypeptides derived from the antibodies are known in the art and are disclosed herein. The monoclonal antibodies of the present invention can be prepared using well-established methods. For example, the monoclonal antibodies can be prepared using hybridoma technology, such as those described by Kohler and Milstein (1975), Nature, 256:495. In a hybridoma method, a mouse, a hamster, or other appropriate host animal, is typically immunized with an immunizing agent (e.g., a cancer cell expressing TfR or extracellular domain and fragments thereof, or TfR or extracellular domain and fragments thereof expressed by the cancer cell) to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-1031). Immortalized cell lines are usually transformed mammalian cells, particularly myeloma cells of rodent, rabbit, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. A signal peptide sequence may be introduced to an antibody or polypeptide provided herein to facilitate the process of producing the antibody or polypeptide. Signal peptide sequence may be any one known in the field, or any of the signal peptide sequences described in the Examples of the present disclosure.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol. (1984), 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies. The antibody may be screened for having specific binding to the epitope on transferrin receptor expressed by the nonhematopoietic cancer or tumor cells, but no specific binding to transferrin receptor expressing leukocytes (e.g., activated T cells), Jurkat cells, and/or other transferrin receptor expressing cells of hematopoietic origin. Cancer cells or extracellular domain (including fragments thereof) containing the epitope may be used for screening.

Jurkat cell line is a lymphoblastoid leukemia cell, and was established from the peripheral blood of a 14 year old boy by Schneider et al. Schneider et al., Int. J. Cancer 19:621-626, 1977. Various Jurkat cell lines are commercially available, for example, from American Type Culture Collection (e.g., ATCC TIB-152, ATCC TIB-153, ATCC CRL-2678).

Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220.

The antibodies identified may further be tested for their capabilities to induce cell death (e.g., apoptosis), and/or inhibiting cell growth or proliferation using methods known in the art and described herein.

After the desired hybridoma cells are identified, the clones may be subcloned by limiting dilution procedures and grown by standard methods (Goding, supra). Suitable culture media for this purpose include, for example, Dulbecco's Modified Eagle's Medium or RPMI-1640 medium. Alternatively, the hybridoma cells may be grown in vivo as ascites in a mammal.

The antibodies or monoclonal antibodies can be generated by culturing the host cells or hybridoma cells, and the antibodies secreted by the host cells or hybridoma cells may further be isolated or purified. Antibodies may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The antibodies of the invention can also be made by recombinant DNA methods, such as those described in U.S. Pat. Nos. 4,816,567 and 6,331,415, which are hereby incorporated by reference. For example, DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiment, the antibodies of the present invention are expressed from two expression vectors. The first expression vector encodes a heavy chain of the antibody, comprising a first part encoding a variable region of the heavy chain of the antibody, and a second part encoding a constant region of the heavy chain of the antibody. In some embodiments, the first part encodes a variable region comprising an amino acid sequence described herein (e.g., amino acids 20-138 of SEQ ID NO:1, amino acids 20-138 of SEQ ID NO:5, amino acids 20-136 of SEQ ID NO:9, amino acids 20-138 of SEQ ID NO:13, or amino acids 1-119 of SEQ ID NO:17). The second expression vector encodes a light chain of the antibody, comprising a first part encoding a variable region of the light chain of the antibody, and a second part encoding a constant region of the light chain of the antibody. In some embodiments, the first part encodes a variable region comprising an amino acid sequence described herein (e.g., amino acids 20-132 of SEQ ID NO:3, amino acids 21-128 of SEQ ID NO:7, amino acids 21-134 of SEQ ID NO:11, amino acids 23-130 of SEQ ID NO:15, or amino acids 1-108 of SEQ ID NO:18).

Alternatively, the antibodies of the present invention are expressed from a single expression vector. The single expression vector encodes both the heavy chain and light chain of the antibodies of the present invention. In some embodiments, the expression vector comprises a polynucleotide sequence encoding a variable region of the heavy chain comprising an amino acid sequence described herein (e.g., amino acids 20-138 of SEQ ID NO:1, amino acids 20-138 of SEQ ID NO:5, amino acids 20-136 of SEQ ID NO:9, amino acids 20-138 of SEQ ID NO:13, or amino acids 1-119 of SEQ ID NO:17) and a variable region of the light chain comprising an amino acid sequence described herein (e.g., amino acids 20-132 of SEQ ID NO:3, amino acids 21-128 of SEQ ID NO:7, amino acids 21-134 of SEQ ID NO:11, amino acids 23-130 of SEQ ID NO:15, or amino acids 1-108 of SEQ ID NO:18).

Normally the expression vector has transcriptional and translational regulatory sequences which are derived from species compatible with a host cell. In addition, the vector ordinarily carries a specific gene(s) which is (are) capable of providing phenotypic selection in transformed cells.

A wide variety of recombinant host-vector expression systems for eukaryotic cells are known and can be used in the invention. For example, Saccharomyces cerevisiae, or common baker's yeast, is the most commonly used among eukaryotic microorganisms, although a number of other strains, such as Pichia pastoris, are available. Cell lines derived from multicellular organisms such as Sp2/0 or Chinese Hamster Ovary (CHO), which are available from the ATCC, may also be used as hosts. Typical vector plasmids suitable for eukaryotic cell transformations are, for example, pSV2neo and pSV2gpt (ATCC), pSVL and pSVK3 (Pharmacia), and pBPV-1/pML2d (International Biotechnology, Inc.).

The eukaryotic host cells useful in the present invention are, preferably, hybridoma, myeloma, plasmacytoma or lymphoma cells. However, other eukaryotic host cells may be suitably utilized provided the mammalian host cells are capable of recognizing transcriptional and translational DNA sequences for expression of the proteins; processing the leader peptide by cleavage of the leader sequence and secretion of the proteins; and providing post-translational modifications of the proteins, e.g., glycosylation.

Accordingly, the present invention provides eukaryotic host cells which are transformed by recombinant expression vectors comprising DNA constructs disclosed herein and which are capable of expressing the antibodies or polypeptides of the present invention. In some embodiments, the transformed host cells of the invention, therefore, comprise at least one DNA construct comprising the light and heavy chain DNA sequences described herein, and transcriptional and translational regulatory sequences which are positioned in relation to the light and heavy chain-encoding DNA sequences to direct expression of antibodies or polypeptides.

The host cells used in the invention may be transformed in a variety of ways by standard transfection procedures well known in the art. Among the standard transfection procedures which may be used are electroporation techniques, protoplast fusion and calcium-phosphate precipitation techniques. Such techniques are generally described by F. Toneguzzo et al. (1986), Mol. Cell. Biol., 6:703-706; G. Chu et al., Nucleic Acid Res. (1987), 15:1311-1325; D. Rice et al., Proc. Natl. Acad. Sci. USA (1979), 79:7862-7865; and V. Oi et al., Proc. Natl. Acad. Sci. USA (1983), 80:825-829.

In the case of two expression vectors, the two expression vectors can be transferred into a host cell one by one separately or together (co-transfer or co-transfect). In some embodiments, the antibody is produced by the host cell. In some embodiments, the antibody is recovered from the culture medium. In some embodiments, the antibody is recovered from the lysed cells.

The present invention also provides a method for producing the antibodies or polypeptides, which comprises culturing a host cell comprising an expression vector(s) encoding the antibodies or the polypeptides, and recovering the antibodies or polypeptides from the culture by ways well known to one skilled in the art.

Furthermore, the desired antibodies can be produced in a transgenic animal. A suitable transgenic animal can be obtained according to standard methods which include micro-injecting into eggs the appropriate expression vectors, transferring the eggs into pseudo-pregnant females and selecting a descendant expressing the desired antibody.

The present invention also provides chimeric antibodies that specifically recognize the epitope on transferrin receptor expressed by a nonhematopoietic cancer cell. For example, the variable and constant regions of the chimeric antibody are from separate species. In some embodiments, the variable regions of both heavy chain and light chain are from the murine antibodies described herein. In some embodiments, the variable regions comprise amino acids 20-138 of SEQ ID NO:1 and/or amino acids 20-132 of SEQ ID NO:3. In some embodiments, the variable regions comprise amino acids 20-138 of SEQ ID NO:5 and/or amino acids 21-128 of SEQ ID NO:7. In some embodiments, the variable regions comprise amino acids 20-136 of SEQ ID NO:9 and/or amino acids 21-134 of SEQ ID NO:11. In some embodiments, the variable regions comprise amino acids 20-138 of SEQ ID NO:13 and/or amino acids 23-130 of SEQ ID NO:15. In some embodiments, the variable regions comprise amino acids 1-119 of SEQ ID NO:17 and/or amino acids 1-108 of SEQ ID NO:18. In some embodiments, the constant regions of both the heavy chain and light chain are from human antibodies. In some embodiments, the constant region is a constant region from human IgG or human IgG1 (such as a constant region described in Table 6). In some embodiments, the chimeric antibody comprises a heavy chain constant region from SEQ ID NO:17 and/or a light chain constant region from SEQ ID NO:18. In some embodiments, the chimeric antibody comprises a heavy chain sequence comprising SEQ ID NO:17 and a light chain sequence comprising SEQ ID NO:18.

The chimeric antibody of the present invention can be prepared by techniques well-established in the art. See for example, U.S. Pat. No. 6,808,901, U.S. Pat. No. 6,652,852, U.S. Pat. No. 6,329,508, U.S. Pat. No. 6,120,767 and U.S. Pat. No. 5,677,427, each of which is hereby incorporated by reference. In general, the chimeric antibody can be prepared by obtaining cDNAs encoding the heavy and light chain variable regions of the antibodies, inserting the cDNAs into an expression vector, which upon being introduced into eukaryotic host cells, expresses the chimeric antibody of the present invention. Preferably, the expression vector carries a functionally complete constant heavy or light chain sequence so that any variable heavy or light chain sequence can be easily inserted into the expression vector.

The present invention provides a humanized antibody that specifically recognizes the epitope on transferrin receptor expressed by a nonhematopoietic cancer cell. The humanized antibody is typically a human antibody in which residues from HVRs or CDRs are replaced with residues from HVRs or CDRs of a non-human species such as mouse, rat or rabbit having the desired specificity, affinity and capacity. In some instances, Fv framework residues of the human antibody are replaced by corresponding non-human residues.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

It is important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the HVR or CDR residues are directly and most substantially involved in influencing antigen binding. The humanized antibodies may also contain modifications in the hinge region to improve one or more characteristics of the antibody.

In another alternative, antibodies may be screened and made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for review see, e.g., Johnson, Kevin S, and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling.” Marks, et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by HVR or CDR grafting, this technique provides completely human antibodies, which have no framework or HVR or CDR residues of rodent origin. It is apparent that although the above discussion pertains to humanized antibodies, the general principles discussed are applicable to customizing antibodies for use, for example, in dogs, cats, primates, equines and bovines.

In certain embodiments, the antibody is a fully human antibody. Non-human antibodies that specifically bind an antigen can be used to produce a fully human antibody that binds to that antigen. For example, the skilled artisan can employ a chain swapping technique, in which the heavy chain of a non-human antibody is co-expressed with an expression library expressing different human light chains. The resulting hybrid antibodies, containing one human light chain and one non-human heavy chain, are then screened for antigen binding. The light chains that participate in antigen binding are then co-expressed with a library of human antibody heavy chains. The resulting human antibodies are screened once more for antigen binding. Techniques such as this one are further described in U.S. Pat. No. 5,565,332. In addition, an antigen can be used to inoculate an animal that is transgenic for human immunoglobulin genes. See, e.g., U.S. Pat. No. 5,661,016.

The antibody may be a bispecific antibody, a monoclonal antibody that has binding specificities for at least two different antigens, can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2 and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

It is contemplated that the present invention encompasses not only the monoclonal antibodies described above, but also any fragments thereof containing the active binding region of the antibodies, such as Fab, F(ab′)₂, scFv, Fv fragments and the like. Such fragments can be produced from the monoclonal antibodies described herein using techniques well established in the art (Rousseaux et al. (1986), in Methods Enzymol., 121:663-69 Academic Press).

Methods of preparing antibody fragment are well known in the art. For example, an antibody fragment can be produced by enzymatic cleavage of antibodies with pepsin to provide a 100 Kd fragment denoted F(ab′)₂. This fragment can be further cleaved using a thiol reducing agent, and optionally a blocking group for the sulfhydryl groups resulting from cleavage of disulfide linkages, to produce 50 Kd Fab′ monovalent fragments. Alternatively, an enzymatic cleavage using papain produces two monovalent Fab fragments and an Fc fragment directly. These methods are described, for example, by U.S. Pat. Nos. 4,036,945 and 4,331,647 and references contained therein, which patents are incorporated herein by reference. Also, see Nisonoff et al. (1960), Arch Biochem. Biophys. 89: 230; Porter (1959), Biochem. J. 73: 119, Edelman et al., in Methods in Enzymology Vol. 1, page 422 (Academic Press 1967).

Alternatively, the Fab can be produced by inserting DNA encoding Fab of the antibody into an expression vector for prokaryote or an expression vector for eukaryote, and introducing the vector into a prokaryote or eukaryote to express the Fab.

The invention encompasses modifications to antibodies or polypeptide described herein, including functionally equivalent antibodies which do not significantly affect their properties and variants which have enhanced or decreased activity and/or affinity. For example, amino acid sequence of an antibody or polypeptide provided herein may be mutated to obtain an antibody with the desired binding affinity to transferrin receptor expressed by the cancer cell. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in the table below under the heading of “conservative substitutions”. If such substitutions result in a change in biological activity, then more substantial changes, denominated “exemplary substitutions” in the table below, or as further described below in reference to amino acid classes, may be introduced and the products screened.

TABLE 1
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Non-polar: Norleucine, Met, Ala, Val, Leu, Ile;
(2) Polar without charge: Cys, Ser, Thr, Asn, Gln;
(3) Acidic (negatively charged): Asp, Glu;
(4) Basic (positively charged): Lys, Arg;
(5) Residues that influence chain orientation: Gly, Pro; and
(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a HVR or CDR domain. In other embodiments, no more than one to three conservative amino acid substitutions are made within a HVR or CDR domain.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g. antibodies, as potential therapeutics is rarely the native cell, variations in the glycosylation pattern of the antibodies can be expected (see, e.g. Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes and the like. Various methods have been proposed to alter the glycosylation pattern achieved in a particular host organism including introducing or overexpressing certain enzymes involved in oligosaccharide production (U.S. Pat. Nos. 5,047,335; 5,510,261 and 5,278,299). Glycosylation, or certain types of glycosylation, can be enzymatically removed from the glycoprotein, for example using endoglycosidase H (Endo H), N-glycosidase F, endoglycosidase F1, endoglycosidase F2, endoglycosidase F3. In addition, the recombinant host cell can be genetically engineered to be defective in processing certain types of polysaccharides. These and similar techniques are well known in the art.

Other methods of modification include using coupling techniques known in the art, including, but not limited to, enzymatic means, oxidative substitution and chelation. Modifications can be used, for example, for attachment of labels for immunoassay. Modified polypeptides are made using established procedures in the art and can be screened using standard assays known in the art.

The antibody or polypeptide of the invention may be conjugated (for example, linked) to an agent, such as a therapeutic agent and a label. Thus, the present disclosure provides a conjugate comprising an antibody or polypeptide described herein and an agent (or therapeutic agent such as a chemotherapeutic agent, cytotoxin, cytotoxic drug, a drug moiety, anticancer agent, or label). Examples of therapeutic agents are radioactive moieties, cytotoxins, cytotoxic drugs, anticancer agent, or chemotherapeutic molecules. Antibody conjugates may be made using methods known in the field, such as the methods described in U.S. Pat. No. 7,553,816, U.S. Pat. No. 6,214,345, and Ducry, L and Stump, B, Bioconjugate chem., 2010, 21: 5-13. In some embodiments, the agent or therapeutic agent is a drug moiety, cytotoxic drug, or cytotoxin described in U.S. provisional application Ser. No. 61/745,448 filed Dec. 21, 2012. In some embodiments, the cytotoxic drug in the conjugate is Dolastatin 10 or a derivative thereof such as Monomethyl Dolastatin 10. In some embodiments, the antibody or polypeptide of the invention is conjugated to an agent, such as a therapeutic agent (e.g., cytotoxic drug or cytotoxin) and a label, through a linker. Thus, the present disclosure provides a conjugate comprising an antibody or polypeptide described herein, an agent (or therapeutic agent, cytotoxin, cytotoxic drug, a drug moiety, or label), and a linker. In some embodiments, the agent or therapeutic agent is a linker described in U.S. provisional application Ser. No. 61/745,448 filed Dec. 21, 2012 or the linker described in example 6 of the present disclosure. Antibody conjugates may be made using the methods described in U.S. provisional application Ser. No. 61/745,448 filed Dec. 21, 2012, the contents of which are incorporated by reference herein in their entirety.

The antibody (or polypeptide) of this invention may be linked to a label such as a fluorescent molecule, a radioactive molecule, an enzyme, or any other labels known in the art. As used herein, the term “label” refers to any molecule that can be detected. In a certain embodiment, an antibody may be labeled by incorporation of a radiolabeled amino acid. In a certain embodiment, biotin moieties that can be detected by marked avidin (e.g., streptavidin containing a fluorescent marker or enzymatic activity that can be detected by optical or colorimetric methods) may be attached to the antibody. In certain embodiments, a label may be incorporated into or attached to another reagent which in turn binds to the antibody of interest. For example, a label may be incorporated into or attached to an antibody that in turn specifically binds the antibody of interest. In certain embodiments, the label or marker can also be therapeutic. Various methods of labeling polypeptides and glycoproteins are known in the art and may be used. Certain general classes of labels include, but are not limited to, enzymatic, fluorescent, chemiluminescent, and radioactive labels. Examples of labels for polypeptides include, but are not limited to, the following: radioisotopes or radionucleoides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., fluorescein isothocyanate (FITC), rhodamine, lanthanide phosphors, phycoerythrin (PE)), enzymatic labels (e.g., horseradish peroxidase, β-galactosidase, luciferase, alkaline phosphatase, glucose oxidase, glucose-6-phosphate dehydrogenase, alcohol dehyrogenase, malate dehyrogenase, penicillinase, luciferase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

The invention also provides pharmaceutical compositions comprising antibodies or polypeptides described herein, and a pharmaceutically acceptable carrier or excipient. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

In some embodiments, the invention provides compositions (described herein) for use in any of the methods described herein, whether in the context of use as a medicament and/or use for manufacture of a medicament.

### Polynucleotides, Vectors and Host Cells

The invention also provides polynucleotides comprising a nucleotide sequence encoding any of the monoclonal antibodies and polypeptides described herein. In some embodiments, the antibodies or polypeptides comprise the sequences of light chain and heavy chain variable regions.

In some embodiments, the polynucleotides comprise a nucleic acid sequence encoding the heavy chain variable region comprising amino acids 20-138 of SEQ ID NO:1 and/or a nucleic acid sequence encoding the light chain variable region comprising amino acids 20-132 of SEQ ID NO:3. In some embodiments, the polynucleotides comprise a nucleic acid sequence encoding the heavy chain variable region comprising amino acids 20-138 of SEQ ID NO:5 and/or a nucleic acid sequence encoding the light chain variable region comprising amino acids 21-128 of SEQ ID NO:7. In some embodiments, the polynucleotides comprise a nucleic acid sequence encoding the heavy chain variable region comprising amino acids 20-136 of SEQ ID NO:9 and/or a nucleic acid sequence encoding the light chain variable region comprising amino acids 21-134 of SEQ ID NO:11. In some embodiments, the polynucleotides comprise a nucleic acid sequence encoding the heavy chain variable region comprising amino acids 20-138 of SEQ ID NO:13 and/or a nucleic acid sequence encoding the light chain variable region comprising amino acids 23-130 of SEQ ID NO:15.

In some embodiments, the polynucleotides comprise a nucleic acid sequence encoding a heavy chain variable region comprising one, two, or three HVRs (or CDRs) from (or of) SEQ ID NO:1, and/or a nucleic acid sequence encoding a light chain variable region comprising one, two, or three HVRs (or CDRs) from (or of) SEQ ID NO:3. In some embodiments, the polynucleotides comprise a nucleic acid sequence encoding a heavy chain variable region comprising one, two, or three HVRs (or CDRs) from (or of) SEQ ID NO:5, and/or a nucleic acid sequence encoding a light chain variable region comprising one, two, or three HVRs (or CDRs) from (or of) SEQ ID NO:7. In some embodiments, the polynucleotides comprise a nucleic acid sequence encoding a heavy chain variable region comprising one, two, or three HVRs (or CDRs) from (or of) SEQ ID NO:9, and/or a nucleic acid sequence encoding a light chain variable region comprising one, two, or three HVRs (or CDRs) from (or of) SEQ ID NO:11. In some embodiments, the polynucleotides comprise a nucleic acid sequence encoding a heavy chain variable region comprising one, two, or three HVRs (or CDRs) from (or of) SEQ ID NO:13, and/or a nucleic acid sequence encoding a light chain variable region comprising one, two, or three HVRs (or CDRs) from (or of) SEQ ID NO:15. In some embodiments, the polynucleotides comprise a nucleic acid sequence encoding a heavy chain variable region comprising one, two, or three HVRs (or CDRs) from (or of) SEQ ID NO:17, and/or a nucleic acid sequence encoding a light chain variable region comprising one, two, or three HVRs (or CDRs) from (or of) SEQ ID NO:18.

In some embodiments, the polynucleotides comprise a nucleic acid sequence comprising nucleotides 58-414 of SEQ ID NO:2, and/or a nucleic acid sequence comprising nucleotides 58-396 of SEQ ID NO:4. In some embodiments, the polynucleotides comprise a nucleic acid sequence comprising nucleotides 58-414 of SEQ ID NO:6, and/or a nucleic acid sequence comprising nucleotides 61-384 of SEQ ID NO:8. In some embodiments, the polynucleotides comprise a nucleic acid sequence comprising nucleotides 58-408 of SEQ ID NO:10, and/or a nucleic acid sequence comprising nucleotides 61-402 of SEQ ID NO:12. In some embodiments, the polynucleotides comprise a nucleic acid sequence comprising nucleotides 58-414 of SEQ ID NO:14, and/or a nucleic acid sequence comprising nucleotides 67-390 of SEQ ID NO:16.

It is appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Thus, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein can, but need not, have an altered structure or function. Alleles can be identified using standard techniques (such as hybridization, amplification and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides can be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston (1994).

The invention also provides vectors (e.g., cloning vectors, expression vectors) comprising a nucleic acid sequence encoding any of the polypeptides (including antibodies) described herein. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides or vectors described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

### Diagnostic Uses

The present invention provides a method of using the antibodies, polypeptides and polynucleotides of the present invention for detection, diagnosis and monitoring of a disease, disorder or condition associated with the epitope expression (either increased or decreased relative to a normal sample, and/or inappropriate expression, such as presence of expression in tissues(s) and/or cell(s) that normally lack the epitope expression).

In some embodiments, the method comprises detecting the epitope expression in a sample obtained from a subject suspected of having cancer, such as pancreatic cancer, gastric cancer, colorectal cancer cells, lung cancer, ovarian cancer, endometrial cancer, prostate cancer, breast cancer, and liver cancer. Preferably, the method of detection comprises contacting the sample with an antibody, polypeptide, or polynucleotide of the present invention and determining whether the level of binding differs from that of a control or comparison sample. The method is also useful to determine whether the antibodies or polypeptides described herein are an appropriate treatment for the patient.

As used herein, the term “a sample” or “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). “A sample” or “a biological sample” further refers to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. Most often, the sample has been removed from an animal, but the term “a sample” or “a biological sample” can also refer to cells or tissue analyzed in vivo, i.e., without removal from animal. Typically, “a sample” or “a biological sample” will contain cells from the animal, but the term can also refer to non-cellular biological material, such as non-cellular fractions of blood, saliva, or urine, that can be used to measure the cancer-associated polynucleotide or polypeptides levels. “A sample” or “a biological sample” further refers to a medium, such as a nutrient broth or gel in which an organism has been propagated, which contains cellular components, such as proteins or nucleic acid molecules.

In one embodiment, the cells or cell/tissue lysate are contacted with an antibody and the binding between the antibody and the cell is determined When the test cells are shown binding activity as compared to a control cell of the same tissue type, it may indicate that the test cell is cancerous. In some embodiments, the test cells are from human tissues.

Various methods known in the art for detecting specific antibody-antigen binding can be used. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (e.g. ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including but not limited to radioisotopes, fluorescent labels, and various enzyme-substrate labels know in the art. Methods of conjugating labels to an antibody are known in the art.

In some embodiments, the polypeptides including antibodies of the invention need not be labeled, and the presence thereof can be detected using a labeled antibody which binds to the antibodies of the invention.

The antibodies of the present invention can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, or ³H) so that the cells or tissue of interest can be localized using immunoscintiography.

The antibody may also be used as staining reagent in pathology using techniques well known in the art.

### Therapeutic Uses

The present invention provides therapeutic uses of the antibodies and polypeptides of the present invention in treating cancer or tumor (such as human cancer or tumor) in an individual. The cancer may be nonhematopoietic cancer, such as, pancreatic cancer, gastric cancer, colorectal cancer cells, lung cancer, ovarian cancer, endometrial cancer, prostate cancer, breast cancer, or liver cancer. In some embodiments, the individual is a human.

The cancer that may be treated using any of the antibodies and polypeptides provided herein includes any of the following: hepatocellular carcinoma, thyroid cancer, colon cancer, colorectal cancer, lung cancer, breast cancer, brain tumor, malignant melanoma, renal cell carcinoma, bladder cancer, lymphomas, T cell lymphomas, multiple myeloma, gastric cancer, pancreas cancer, cervical cancer, endometrial carcinoma, ovarian cancer, endometrial cancer, esophageal cancer, liver cancer, head and neck squamous cell carcinoma, cutaneous cancer, urinary tract carcinoma, prostate cancer, choriocarcinoma, pharyngeal cancer, laryngeal cancer, thecomatosis, androblastoma, endometrium hyperplasy, endometriosis, embryoma, fibrosarcoma, Kaposi's sarcoma, hemangioma, cavernous hemangioma, angioblastoma, retinoblastoma, astrocytoma, neurofibroma, oligodendroglioma, medulloblastoma, ganglioneuroblastoma, glioma, rhabdomyosarcoma, hamartoblastoma, osteogenic sarcoma, leiomyosarcoma, thyroid sarcoma and Wilms tumor, as long as the cancer cell expresses the epitope recognized by the antibodies or the polypeptides described herein.

The cancer that may be treated using any of the antibodies and polypeptides provided herein may also be any of the following: adrenal gland tumors, AIDS-associated cancers, alveolar soft part sarcoma, astrocytic tumors, bladder cancer (squamous cell carcinoma and transitional cell carcinoma), bone cancer (adamantinoma, aneurysmal bone cysts, osteochondroma, osteosarcoma), brain and spinal cord cancers, metastatic brain tumors, breast cancer, carotid body tumors, cervical cancer, chondrosarcoma, chordoma, chromophobe renal cell carcinoma, clear cell carcinoma, colon cancer, colorectal cancer, cutaneous benign fibrous histiocytomas, desmoplastic small round cell tumors, ependymomas, Ewing's tumors, extraskeletal myxoid chondrosarcoma, fibrogenesis imperfecta ossium, fibrous dysplasia of the bone, gallbladder and bile duct cancers, gestational trophoblastic disease, germ cell tumors, head and neck cancers, islet cell tumors, Kaposi's sarcoma, kidney cancer (nephroblastoma, papillary renal cell carcinoma), leukemias, lipoma/benign lipomatous tumors, liposarcoma/malignant lipomatous tumors, liver cancer (hepatoblastoma, hepatocellular carcinoma), lymphomas, lung cancer, medulloblastoma, melanoma, meningiomas, multiple endocrine neoplasia, multiple myeloma, myelodysplastic syndrome, neuroblastoma, neuroendocrine tumors, ovarian cancer, endometrial cancer, pancreatic cancers, papillary thyroid carcinomas, parathyroid tumors, pediatric cancers, peripheral nerve sheath tumors, phaeochromocytoma, pituitary tumors, prostate cancer, uveal or intraocular melanoma, rare hematologic disorders, renal metastatic cancer, rhabdoid tumor, rhabdomysarcoma, sarcomas, skin cancer, soft-tissue sarcomas, squamous cell cancer, stomach cancer, synovial sarcoma, testicular cancer, thymic carcinoma, thymoma, thyroid metastatic cancer, and uterine cancers (carcinoma of the cervix, endometrial carcinoma, and leiomyoma). The cancer cell described herein may express the epitope recognized by the antibodies or the polypeptides described herein.

The method may further comprise a step of detecting the binding between an antibody or a polypeptide described herein and a tumor or cancer cell in an individual to be treated.

Generally, an effective amount of a composition comprising an antibody or a polypeptide is administered to a subject in need of treatment, thereby inhibiting growth of the cancer cell and/or inducing death of the cancer cell.

Also provided herein are methods of treating nonhematopoietic cancer in an individual comprising administering to the individual an antibody or polypeptide provided herein and another anti-cancer agent. In some embodiments, the antibody and the anti-cancer agent in conjunction provide effective treatment of cancer in the individual. The antibody provided herein and the other anti-cancer agent may be in separate compositions or in same composition. The antibody provided herein and the other anti-cancer agent may be in separate administrations, in simultaneous administration, or in sequential administrations.

Any of the compositions provided herein may be formulated with a pharmaceutically acceptable carrier. In one embodiment, the composition is formulated for administration by intraperitoneal, intravenous, subcutaneous, and intramuscular injections, and other forms of administration such as oral, mucosal, via inhalation, sublingually, etc.

In another embodiment, the present invention also contemplates administration of a composition comprising the antibodies or polypeptides of the present invention conjugated to other molecules, such as detectable labels, agents, therapeutic agents (e.g., chemotherapeutic agents), drug moieties, anticancer agents, or cytotoxic agents (such as cytotoxin). In some embodiments, the antibodies or polypeptides of the present invention is conjugated through a linker to other molecules, such as detectable labels, agents, therapeutic agents, drug moieties, or cytotoxic agents (such as cytotoxin). The agents may include, but are not limited to radioisotopes, toxins, toxoids, inflammatory agents, enzymes, antisense molecules, peptides, cytokines, or chemotherapeutic agents. Methods of conjugating the antibodies with such molecules are generally known to those of skilled in the art. See, e.g., PCT publications WO 92/08495; WO 91/14438; WO 89/12624; U.S. Pat. No. 5,314,995; and EP 396,387; the disclosures of which are incorporated herein by reference in their entireties. Antibody conjugates may also be made using methods described in U.S. provisional application Ser. No. 61/745,448 filed Dec. 21, 2012.

In one embodiment, the composition comprises an antibody or polypeptide conjugated to a cytotoxic agent. Cytotoxic agents can include any agents that are detrimental to cells. A preferred class of cytotoxic agents that can be conjugated to the antibodies or fragments may include, but are not limited to paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, Dolastatin 10 or a derivative thereof such as Monomethyl Dolastatin 10, and puromycin and analogs or homologs thereof.

The dosage required for the treatment depends on the choice of the route of administration, the nature of the formulation, the nature of the subject's illness, the subject's size, weight, surface area, age and sex; other drugs being administered, and the judgment of the attending physician. Suitable dosages are in the range of 0.01-1000.0 mg/kg.

Generally, any of the following doses may be used: a dose of at least about 50 mg/kg body weight; at least about 10 mg/kg body weight; at least about 3 mg/kg body weight; at least about 1 mg/kg body weight; at least about 750 μg/kg body weight; at least about 500 μg/kg body weight; at least about 250 μg/kg body weight; at least about 100 μg/kg body weight; at least about 50 μg/kg body weight; at least about 10 μg/kg body weight; at least about 1 μg/kg body weight, or less, is administered. For repeated administrations over several days or longer, depending on the condition, the treatment is sustained until a desired suppression of disease symptoms occurs. An exemplary dosing regimen comprises administering a weekly dose of about 6 mg/kg of the antibody. However, other dosage regimens may be useful, depending on the pattern of pharmacokinetic decay that the practitioner wishes to achieve. Empirical considerations, such as the half-life, generally will contribute to determination of the dosage. The progress of this therapy is easily monitored by conventional techniques and assays.

In some subjects, more than one dose may be required. Frequency of administration may be determined and adjusted over the course of therapy. For example, frequency of administration may be determined or adjusted based on the type and stage of the cancer to be treated, whether the agent is administered for preventive or therapeutic purposes, previous therapy, the patient's clinical history and response to the agent, and the discretion of the attending physician. Typically the clinician will administer a therapeutic antibody, until a proper dosage is reached to achieve the desired result. In some cases, sustained continuous release formulations of antibodies may be appropriate. Various formulations and devices for achieving sustained release are known in the art.

In one embodiment, dosages for the antibodies or polypeptides may be determined empirically in subjects who have been given one or more administration(s). Subjects are given incremental dosages of the antibodies or polypeptides. To assess efficacy of the antibodies or polypeptides, markers of the disease symptoms such as transferrin receptor can be monitored. Efficacy in vivo can also be measured by assessing tumor burden or volume, the time to disease progression (TDP), and/or determining the response rates (RR).

Administration of an antibody or polypeptide in accordance with the method in the present invention can be continuous or intermittent, depending, for example, upon the recipient's physiological condition, whether the purpose of the administration is therapeutic or prophylactic, and other factors known to skilled practitioners. The administration of an antibody or a polypeptide may be essentially continuous over a preselected period of time or may be in a series of spaced dose.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

In another embodiment, the composition can comprise one or more anti-cancer agents, one or more antibodies described herein, or with an antibody or polypeptide that binds to a different antigen. Such composition can contain at least one, at least two, at least three, at least four, at least five different antibodies. The antibodies and other anti-cancer agents may be in the same formulation (e.g., in a mixture, as they are often denoted in the art), or in separate formulations but are administered concurrently or sequentially, are particularly useful in treating a broader range of population of individuals.

A polynucleotide encoding any of the antibodies or polypeptides of the present invention can also be used for delivery and expression of any of the antibodies or polypeptides of the present invention in a desired cell. It is apparent that an expression vector can be used to direct expression of the antibody or polypeptide. The expression vector can be administered by any means known in the art, such as intraperitoneally, intravenously, intramuscularly, subcutaneously, intrathecally, intraventricularly, orally, enterally, parenterally, intranasally, dermally, sublingually, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding any of the antibodies or polypeptides of the present invention can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al. (1990), Proc. Natl. Acad. Sci. USA, 87:3655; Wu et al. (1991), J. Biol. Chem. 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol.

The therapeutic polynucleotides and polypeptides of the present invention can be delivered using gene delivery vehicles. The gene delivery vehicle can be of viral or non-viral origin (see generally, Jolly (1994), Cancer Gene Therapy 1:51; Kimura (1994), Human Gene Therapy 5:845; Connelly (1985), Human Gene Therapy 1:185; and Kaplitt (1994), Nature Genetics 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP Patent No. 0 345 242; alphavirus-based vectors, e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors, e.g., PCT Publication Nos. WO 94/12649, WO 93/03769; WO 93/19191; WO 94/28938; WO 95/11984 and WO 95/00655. Administration of DNA linked to killed adenovirus as described in Curiel (1992), Hum. Gene Ther. 3:147 can also be employed.

Non-viral delivery vehicles and methods can also be employed, including, but are not limited to, polycationic condensed DNA linked or unlinked to killed adenovirus alone (see, e.g., Curiel (1992), Hum. Gene Ther. 3:147); ligand-linked DNA (see, e.g., Wu (1989), J. Biol. Chem. 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes.

Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent NO. 0 524 968. Additional approaches are described in Philip (1994), Mol. Cell. Biol. 14:2411 and in Woffendin (1994), Proc. Natl. Acad. Sci. 91:1581.

The composition comprising an antibody of the present invention can be administered (e.g., administered sequentially or concurrently) with one or more other therapeutic agents such as chemotherapeutic agents (such as 5-FU, 5-FU/MTX, 5-FU/Leucovorin, Levamisole, Irinotecan, Oxaliplatin, Capecitabin, or Uracil/Tegafur), immunoadjuvants, growth inhibitory agents, cytotoxic agents and cytokines, etc. The amounts of the antibody and the therapeutic agent depend on what type of drugs are used, the pathological condition being treated, and the scheduling and routes of administration but would generally be less than if each were used individually.

Following administration of the composition comprising the antibody described herein, the efficacy of the composition can be evaluated both in vitro and in vivo by various methods well known to one of ordinary skill in the art. Various animal models are well known for testing anti-cancer activity of a candidate composition. These include human tumor xenografting into athymic nude mice or scid/scid mice, or genetic murine tumor models such as p53 knockout mice. The in vivo nature of these animal models make them particularly predictive of responses in human patients. Such models can be generated by introducing cells into syngeneic mice using standard techniques, e.g., subcutaneous injection, tail vein injection, spleen implantation, intraperitoneal implantation and implantation under the renal capsule, etc.

### Kits

The invention also provides kits for use in the instant methods. Kits of the invention include one or more containers comprising an antibody or a polypeptide (e.g., purified or isolated antibody or polypeptide) described herein and instructions for use in accordance with any of the methods of the invention described herein. In some embodiments, these instructions comprise a description of administration of the antibody or polypeptide to treat a nonhematopoietic cancer (such as pancreatic cancer, gastric cancer, colorectal cancer cells, lung cancer, ovarian cancer, endometrial cancer, prostate cancer, breast cancer, and liver cancer), according to any of the methods described herein. In some embodiments, these instructions comprise administering an antibody or a polypeptide provided herein and another anti-cancer agent for treating a nonhematopoietic cancer, whereby the antibody and the anti-cancer agent in conjunction provide effective treatment of cancer. The kit may further comprise a description of selecting an individual suitable for treatment based on identifying whether that individual has the disease and the stage of the disease, or whether the individual has cancer or tumor expressing transferrin receptor to which an antibody or polypeptide provided herein binds or recognizes. In some embodiments, the kits further comprise a second anti-cancer agent.

In some embodiments, the kits for detecting a cancer cell in a sample comprise an antibody or a polypeptide described herein and/or reagents for detecting binding of the antibody or the polypeptide to a cell in the sample.

The instructions relating to the use of the antibodies or polypeptides to treat cancer generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for treating a cancer described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody described herein. The container may further comprise a second pharmaceutically active agent.

Kits may optionally provide additional components such as buffers and interpretive information. Normally, the kit comprises a container and a label or package insert(s) on or associated with the container.

The following are examples of the methods and compositions of the invention. It is understood that various other embodiments may be practiced, given the general description provided above.

### EXAMPLES

### Example 1

### Generation of Cancer Specific Transferrin Receptor Antibodies

Balb/c mice were immunized and boosted three times with 300 μg of pancreatic cancer cell line Panc 02.03B membrane fraction or 50 μg of transferrin receptor purified from Panc 02.03B. Spleen cells were fused with P3X63 myeloma cells to generate hybridomas that were then cultured in DMEM supplemented with 10% FBS (Hyclone®) and HAT (Hybri-Max®, Sigma H0262, at a final concentration of 100 μM hypoxanthine, 0.4 μM aminopterin, 16 μM thymidine). Hybridomas were screened for positive binding to cancer-derived transferrin receptor, or cancer cells, but negative to activated T cells. Screening of hybridomas that were positive for binding to Panc 02.03B cancer cells but negative for binding to activated T cells resulted in isolation of three hybridoma clones, 6-90 (IgM, κ), 55-31 (IgM, κ) and 122-72 (IgM, κ).

An additional hybridoma was produced and isolated by immunizing and providing a one-time boost to Balb/c mice with 300 μg of membrane fraction from the lung cancer cell line H358. Spleen cells were fused with P3X63 myeloma cells to generate hybridomas for selection as described above and antibodies produced by hybridomas were screened for positive H358 binding by FACS. One hybridoma clone 5D7-54.17, identified as mouse IgM, K, was selected due to its ability to recognize the human transferrin receptor on the lung cancer cell line H358.

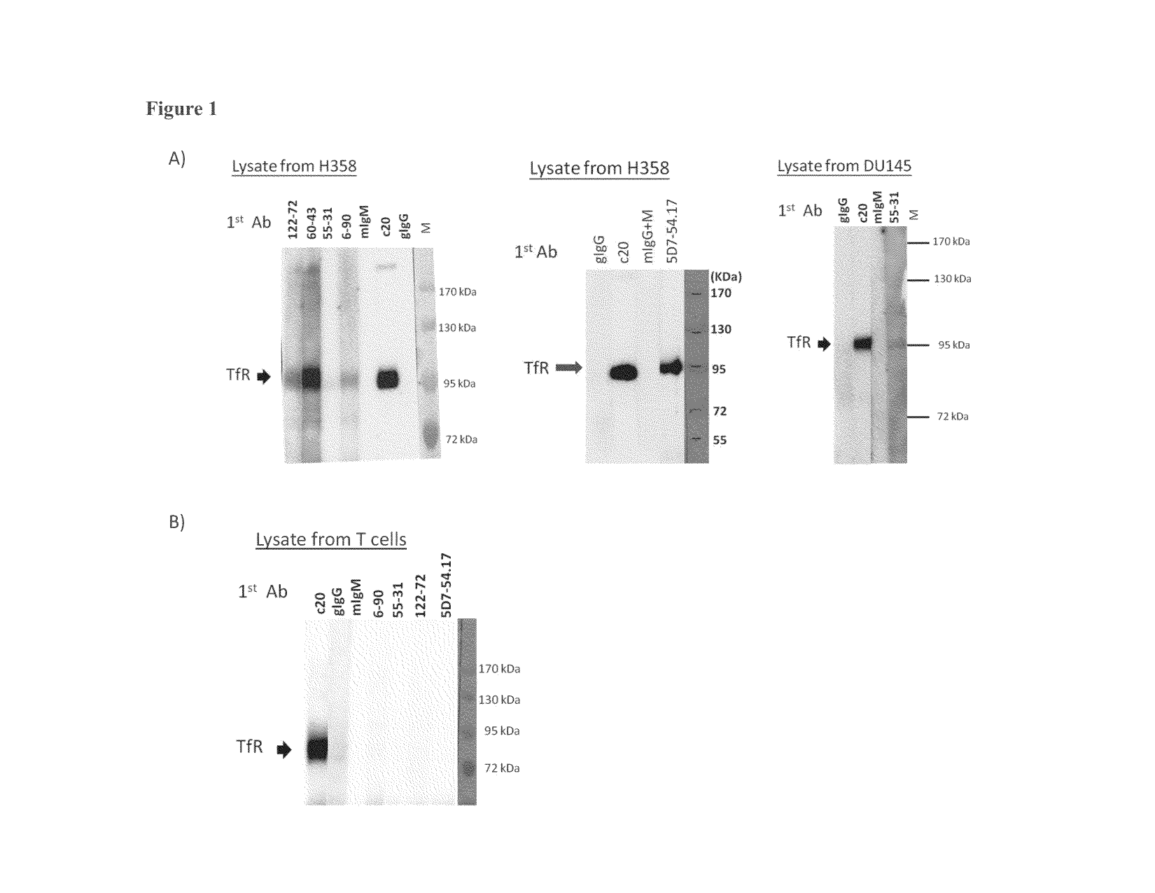

Immunoprecipitation and western blot analyses were conducted to further demonstrate their specificity to transferrin receptor expressed by cancer cells (FIG. 1). Cell lysates from lung cancer H358 or prostate cancer DU145 cell lines were immunoprecipitated with anti-human transferrin receptor antibody, MEM189. Another commercially available anti-human transferrin receptor Ab, C20, was used as positive control to locate the position of human transferrin receptor (˜95 kDa) on SDS-PAGE. FIG. 1A shows that MEM189-immunoprecipitated protein from H358 or DU145 can be recognized by 6-90, 122-72, 5D7-54.17 or 55-31, respectively. Antibody 60-43 had the same CDRs as antibody 122-72. In contrast, the MEM189-immunoprecipitated protein prepared from lysate of activated T cells can be recognized only by C20 (FIG. 1B), but not by these four antibodies. These data suggest that these four antibodies were able to recognize cancer-specific modifications of transferrin receptor but not the protein backbone or modification occurring in normal cells such as activated T cells.

To determine the amino acid and nucleotide sequences of the antibodies, the cDNAs of the antibody light and heavy chain variable regions were amplified by PCR, and the synthesized cDNAs were subcloned into pCRII (Invitrogen™) for sequence determination. Nucleotide sequences from several independent clones were analyzed. The nucleotide sequences of the variable region of heavy and light chains including the signal peptide, and the translated protein sequences are shown in FIGS. 2-5. The signal peptides used in the heavy chain and light chain for the antibodies are indicated in these figures. The Kabat CDRs for these antibodies are bolded and underlined in these figures. Constant region sequences of mouse immunoglobulin M heavy chain (Kawakami et al. Nucleic Acids Res. 8(17): 3933-3945, 1980) and Kappa light chain (Hieter et al. Cell 22(1 Pt 1):197-207, 1980) isotype were previously described.

### Example 2

### Binding of Anti-Transferrin Receptor Antibodies to Normal and Cancer Cells

The binding of 6-90, 55-31, 122-72 and 5D7-54.17 anti-transferrin receptor antibodies to human normal cells, including red blood cells, polymorphonuclear leukocytes (PMN), lymphocytes, monocytes, platelets, and Human Umbilical Vein Endothelial Cells (HUVEC), was examined by FACS analysis. The hyper-immuned serum (Hyp-serum) or the corresponding cell marker antibodies for certain cell types were included as positive controls. Table 2 shows that 6-90, 55-31 and 122-72 did not bind to normal cells when compared to mouse IgM isotype control. Positive binding to lymphocytes (ranging from 4 to 42%) was detected by 5D7-54.17, yet further tests showed that it was not able to bind to T lymphocytes and was only able to bind minimally to B lymphocytes (data not shown). Compared to the other three antibodies, slight binding (ranging from 8-19%) of 5D7-54.17 to PMN and monocytes of certain donors was observed.

TABLE 2
Binding profile of 6-90, 55-31 122-72 and 5D7-54.17 anti-transferrin receptor
antibodies to normal cells
Cell type: RBC*

| | | | 6-90 | | 55-31 | | 122-72 | | 5D7-54.17 | |
|---|---|---|---|---|---|---|---|---|---|---|
| (%) | mIgM | Hyp-serum | 1x | 1/10x | 1x | 1/10x | 1x | 1/10x | 1x | 1/10x |
| D102 | <1 | 10 | <1 | <1 | <1 | <1 | <1 | <1 | 1 | <1 |
| D005 | <1 | 59 | <1 | <1 | <1 | <1 | <1 | <1 | <1 | <1 |
| D014 | <1 | 76 | 1 | <1 | 1 | 1 | 1 | 1 | 3 | 2 |
| D016 | <1 | 54 | 1 | 1 | 1 | 1 | 1 | 1 | 3 | 2 |

Cell Type: PMN*

| | | | | 6-90 | | 55-31 | | 122-72 | | 5D7-54.17 | |
|---|---|---|---|---|---|---|---|---|---|---|---|
| (%) | mIgM | Hyp-serum | Anti-sLeX | 1x | 1/10x | 1x | 1/10x | 1x | 1/10x | 1x | 1/10x |
| D102 | <1 | 100 | 100 | 1 | 1 | 1 | 1 | 1 | 1 | 3 | 1 |
| D005 | <1 | 99 | 98 | 1 | <1 | 1 | 1 | <1 | <1 | 12 | 2 |
| D014 | <1 | 99 | 98 | <1 | <1 | <1 | <1 | 1 | <1 | 8 | 9 |
| D016 | <1 | 97 | 87 | 1 | <1 | <1 | <1 | <1 | 1 | 9 | 8 |

| | | | 6-90 | | 55-31 | | 122-72 | | 5D7-54.17 | |
|---|---|---|---|---|---|---|---|---|---|---|
| (%) | mIgM | Hyp-serum | 1x | 1/10x | 1x | 1/10x | 1x | 1/10x | 1x | 1/10x |

Cell Type: Lymphocytes*

| | | | | | | | | | | |
|---|---|---|---|---|---|---|---|---|---|---|
| D102 | 3 | 40 | <1 | 2 | <1 | 1 | <1 | 2 | 5 | 4 |
| D005 | 4 | 37 | <1 | 3 | <1 | 2 | <1 | 1 | 3 | 4 |
| D014 | 1 | 77 | 1 | 1 | 1 | 1 | <1 | 1 | 10 | 26 |
| D016 | 1 | 78 | 1 | 1 | 1 | 1 | <1 | 1 | 12 | 42 |

Cell Type: Monocytes*

| | | | | | | | | | | |
|---|---|---|---|---|---|---|---|---|---|---|
| D102 | 2 | 96 | 2 | 2 | 3 | 1 | 1 | 4 | 3 | 3 |
| D005 | 3 | 93 | 2 | 4 | 4 | 3 | 2 | 4 | 12 | 12 |
| D014 | <1 | 85 | <1 | <1 | <1 | <1 | <1 | 1 | 4 | 19 |
| D016 | 1 | 82 | <1 | <1 | 1 | <1 | <1 | 1 | 3 | 7 |

Cell type: Platelets*

| | | | 6-90 | | 55-31 | | 122-72 | | 5D7-54.17 | |
|---|---|---|---|---|---|---|---|---|---|---|
| (%) | mIgM | Anti-CD41a | 1x | 1/10x | 1x | 1/10x | 1x | 1/10x | 1x | 1/10x |
| D102 | <1 | 78 | <1 | <1 | <1 | <1 | <1 | <1 | 1 | 1 |
| D005 | <1 | 79 | <1 | <1 | <1 | <1 | <1 | <1 | 1 | <1 |
| D014 | <1 | 97 | <1 | <1 | <1 | <1 | <1 | <1 | 2 | 4 |
| D016 | <1 | 95 | <1 | <1 | <1 | <1 | <1 | <1 | 2 | 6 |

Cell Type: HUVEC*

| mIgM | Anti-CD31 | 6-90 | | 55-31 | | 122-72 | | 5D7-54.17 | |
|---|---|---|---|---|---|---|---|---|---|
| 5 ug/ml | (3 ug/ml) | 1x | 1/10x | 1x | 1/10x | 1x | 1/10x | 1x | 1/10x |
| 1 | 99 | 1 | 1 | 2 | 3 | 1 | 1 | 2 | 2 |

*Numbers indicate % positive cell binding

The binding of 6-90, 55-31, 122-72 and 5D7-54.17 anti-transferrin receptor antibodies to human cancer cell lines, including lung, pancreatic, gastric, ovarian, endometrial, colorectal, breast, prostate and liver cancer cells, was examined. The data are summarized in Table 3. 6-90 was shown to bind strongly (mean fluorescence intensity >1000) to H358, Panc 02.03B, SU.86.86, SNU-16, NCI-N87, Kato III, OMC-3, DLD-1, Colo 205 and WiDr. 122-72 was able to bind strongly (mean fluorescence intensity >1000) to H358, Panc 02.03B, SU.86.86, SNU-16, NCI-N87, OMC-3, DLD-1, Colo 205 and WiDr. 5D7-54.17 was shown to bind strongly (mean fluorescence intensity >1000) to H358, Panc 02.03B, SU.86.86, SNU-16, NCI-N87, Kato III, OMC-3, SK-OV-3, DLD-1, Colo 205, WiDr, MDA-MB-453 and KLE. 55-31 was shown to bind A549, Panc 02.03B, Panc-1, SK-OV-3, DU-145 and HEC-1-A. These antibodies did not bind to leukemia cell line, Jurkat. The binding intensity (mean fluorescence intensity) of isotype control was mostly under 10, except in NCI-N87 (MFI: 33), OMC-3 cells (MFI: 16), KLE(MFI:10) and HEC-1-A (MFI: 13).

Taken together, 6-90, 55-31, 122-72 and 5D7-54.17 anti-transferrin receptor antibodies were able to bind to a broad spectrum of human cancer cells, including colorectal, gastric, pancreatic, ovarian, endometrial, lung cancers, prostate, breast, and liver cancers.

TABLE 3
Binding profile of 6-90, 55-31 122-72 and 5D7-54.17 anti-transferrin receptor
antibodies to cancer cells (cancer cell lines with binding MFI > 50 are shown)
6-90*

| | Isotype control |
|---|---|
| 6-90 | (IgM) |

| Cancer type | Cell line | 1X | 1/10X | 5 ug |
|---|---|---|---|---|
| Lung cancer | H358 | 823 | 1597 | 4 |

| | |
|---|---|
| | Isotype control |
| 6-90 | (IgM) |

| Cancer type | Cell line | 1X | ⅕X | 5 ug |
|---|---|---|---|---|
| Pancreatic cancer | Panc02.03B | 5913 | 4907 | 5 |
| | SU.86.86 | 3948 | 2936 | 6 |
| Gastric cancer | SNU-16 | 4752 | 3971 | 5 |
| | NCI-N87 | 5811 | 5095 | 33 |
| | Kato III | 1409 | 1246 | 7 |
| Ovarian cancer | OMC-3 | 3139 | 2149 | 16 |
| Colorectal cancer | DLD-1 | 3765 | 3905 | 5 |
| | COLO205 | 7092 | 6158 | 3 |
| | WiDr | 1423 | 1085 | 5 |
| Breast cancer | MDA-MB-453 | 147 | 141 | 4 |
| Endometrial | HEC-1-A | 57 | 11 | 13 |
| cancer | KLE | 890 | 556 | 10 |

55-31*

| | |
|---|---|
| | Isotype control |
| 55-31 | (IgM) |

| Cancer type | Cell line | 1X | 1/10X | 5 ug |
|---|---|---|---|---|
| Lung cancer | H358 | 234 | 295 | 4 |

| | |
|---|---|
| | Isotype control |
| 55-31 | (IgM) |

| Cancer type | Cell line | 1X | ⅕X | 5 ug |
|---|---|---|---|---|
| Lung cancer | A549 | 897 | 711 | 8 |
| Pancreatic cancer | Panc02.03B | 574 | 314 | 5 |
| | SU.86.86 | 154 | 96 | 6 |
| | Panc-1 | 773 | 503 | 6 |
| Gastric cancer | Kato III | 140 | 97 | 7 |
| Ovarian cancer | OMC-3 | 214 | 179 | 16 |
| | SK-OV-3 | 2369 | 1973 | 5 |
| Colorectal cancer | DLD-1 | 219 | 104 | 5 |
| Breast cancer | MDA-MB-453 | 81 | 65 | 4 |
| Prostate cancer | DU145 | 2285 | 2276 | 4 |
| Endometrial | HEC-1-A | 1491 | 1237 | 13 |
| cancer | KLE | 136 | 87 | 10 |

122-72*

| | |
|---|---|
| | Isotype control |
| 122-72 | (IgM) |

| Cancer type | Cell line | 1X | 1/10X | 5 ug |
|---|---|---|---|---|
| Lung cancer | H358 | 942 | 2456 | 4 |

| | |
|---|---|
| | Isotype control |
| 122-72 | (IgM) |

| Cancer type | Cell line | 1x | ⅕X | 5 ug |
|---|---|---|---|---|
| Lung cancer | H727 | 623 | 319 | 9 |
| Pancreatic cancer | Panc02.03B | 4689 | 4666 | 5 |
| | SU.86.86 | 5073 | 4600 | 6 |
| Gastric cancer | SNU-16 | 4149 | 5095 | 5 |
| | NCI-N87 | 3937 | 4296 | 33 |
| | Kato III | 613 | 724 | 7 |
| Ovarian cancer | OMC-3 | 3459 | 3043 | 16 |
| | SK-OV-3 | 467 | 381 | 5 |
| Colorectal cancer | DLD-1 | 2498 | 2693 | 5 |
| | COLO205 | 6661 | 6342 | 3 |
| | WiDr | 2475 | 2460 | 5 |
| Breast cancer | MDA-MB-453 | 53 | 16 | 4 |
| Prostate cancer | DU145 | 51 | 13 | 4 |
| Endometrial | HEC-1-A | 72 | 15 | 13 |
| cancer | | | | |

5D7-54.17*

| | |
|---|---|
| | Isotype control |
| 5D7-54.17 | (IgM) |

| Cancer type | Cell line | 1X | 1/10X | 5 ug |
|---|---|---|---|---|
| Lung cancer | H358 | 1811 | 2849 | 4 |

| | |
|---|---|
| | Isotype control |
| 5D7-54.17 | (IgM) |

| Cancer type | Cell line | 1X | ⅕X | 5 ug |
|---|---|---|---|---|
| Lung cancer | H520 | 64 | 26 | 5 |
| | H727 | 427 | 56 | 9 |
| Pancreatic cancer | Panc02.03B | 5346 | 4617 | 5 |
| | SU.86.86 | 5408 | 3535 | 6 |
| | Panc-1 | 67 | 40 | 6 |
| Gastric cancer | SNU-16 | 6360 | 6657 | 5 |
| | NCI-N87 | 6883 | 7444 | 33 |
| | Kato III | 2419 | 2096 | 7 |
| Ovarian cancer | OMC-3 | 5650 | 4843 | 16 |
| | SK-OV-3 | 1342 | 757 | 5 |
| Colorectal cancer | DLD-1 | 3982 | 4583 | 5 |
| | COLO205 | 6313 | 5359 | 3 |
| | WiDr | 2908 | 2649 | 5 |
| Breast cancer | MDA-MB-453 | 1146 | 1153 | 4 |
| Prostate cancer | DU145 | 81 | 23 | 4 |
| | PC3 | 81 | 27 | 3 |
| | 22Rv1 | 51 | 24 | 7 |
| Liver cancer | Hep G2 | 122 | 106 | 4 |
| | Hep 3B2.1-7 | 441 | 419 | 5 |
| Endometrial | HEC-1-A | 294 | 273 | 13 |
| cancer | KLE | 1340 | 1440 | 10 |

Cell type: T Leukemia**

| | Isotype | Isotype | | | | | | | | | |
|---|---|---|---|---|---|---|---|---|---|---|---|
| | control | control | | | | | | | | | |
| | IgM | IgG | MEM189 | 6-90 | | 55-31 | | 122-72 | | 5D7-54.17 | |
| | 5 ug | 1 ug | 1 ug | 1X | 1/10X | 1X | 1/10X | 1X | 1/10X | 1X | 1/10X |
| Jurkat | <1 | <1 | 100 | <1 | <1 | 1 | <1 | 1 | <1 | 2 | <1 |

*Numbers indicate mean fluorescence intensity, MFI.
**Numbers indicate % of gated cells

### Example 3

### Induction of Apoptosis by Anti-Transferrin Receptor Antibodies

The 6-90, 55-31, 122-72 and 5D7-54.17 anti-transferrin receptor antibodies were shown above to bind to a variety of human cancer cell lines. The antibodies were then examined for their apoptosis-inducing capacity in the cancer cell lines that demonstrated positive binding by the antibodies. Staining of Annexin V and PI, as detected by FACS analysis, was performed to measure cell apoptosis induced by these antibodies. Cells were incubated in the presence of hybridoma culture medium (titrated dilution indicated) at 37° C. for overnight. Table 4 summarizes the cell lines in which the antibodies induced 10-60% apoptosis over the background (signal induced by the isotype control mouse IgM).

TABLE 4
Summary of apoptosis-inducing capacity of cancer-
specific anti-transferrin receptor antibodies.

| Antibody | Cells | 1/2X | 1/6X | 1/18X | IgM |
|---|---|---|---|---|---|
| 6-90 | DLD1 | 50.6 | 42.3 | 38.9 | 24.3 |
| | COLO205 | 50.9 | 42.2 | 22.9 | 19.1 |
| | H358 | 50.7 | 49.0 | 46.9 | 31.8 |
| | SNU-16 | 40.2 | 37.2 | 34.8 | 30.0 |
| | Panc 02.03B | 46.7 | 28.7 | 24.5 | 19.0 |
| 55-31 | DU145 | 31.0 | 35.4 | 23.6 | 19.5 |
| 122-72 | DLD1 | 42.2 | 38.6 | 37.1 | 24.3 |
| | COLO205 | 80.3 | 25.2 | 18.6 | 19.1 |
| | H358 | 70.8 | 70.1 | 63.9 | 31.8 |
| | SNU-16 | 46.6 | 43.5 | 36.6 | 30.0 |
| | Pan 02.03B | 31.5 | 20.5 | 20.5 | 16.9 |
| | SU.86.86 | 41.3 | 29.3 | 23.4 | 12.1 |
| 5D7-54.17 | DLD1 | 44.4 | 44.4 | 42.1 | 24.3 |
| | COLO205 | 59.5 | 44.4 | 21.7 | 19.1 |
| | H358 | 55.9 | 48.6 | 37.7 | 31.8 |
| | SNU-16 | 57.3 | 50.7 | 42.8 | 30.0 |
| | Pan 02.03B | 44.0 | 41.0 | 27.0 | 16.9 |

Numbers indicate the percentage of Annexin V positive and/or PI positive cells

### Example 4

### Internalization Assay

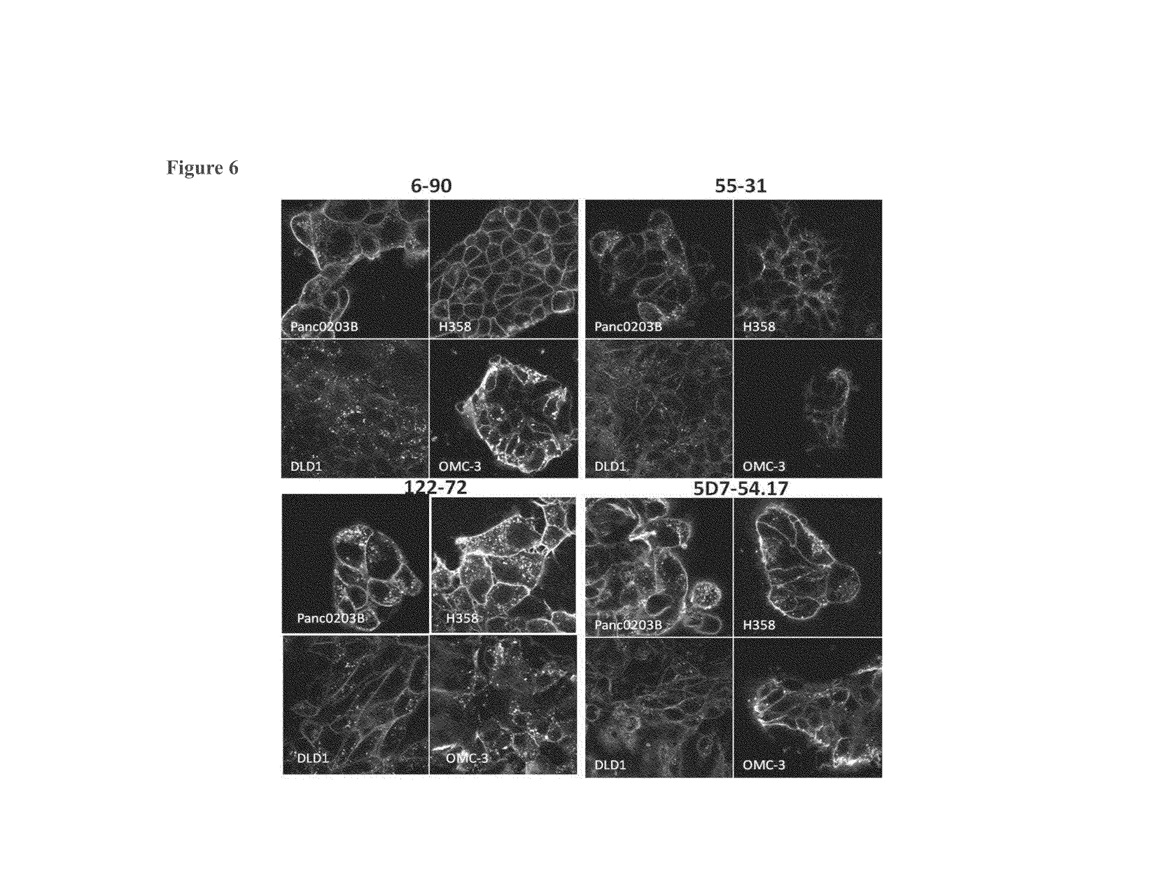

6-90, 55-31, 122-72 and 5D7-54.17 antibodies were tested for their ability to induce receptor internalization in binding-positive cancer cell lines such as Panc 02.03B, H358, DLD-1 and OMC-3. Cancer cells were treated with antibodies at 37° C. for 4 hours, and internalization was detected by the fluorescence-labeled secondary antibody. As shown in FIG. 6, 6-90, 55-31, 122-72 and 5D7-54.17 were found to accumulate in the cytosol of cancer cells after incubation at 37° C. In contrast, incubation at 4° C. did not induce internalization and only membrane staining was observed (data not shown).

### Example 5

### Determination of Cancer Derived Epitopes

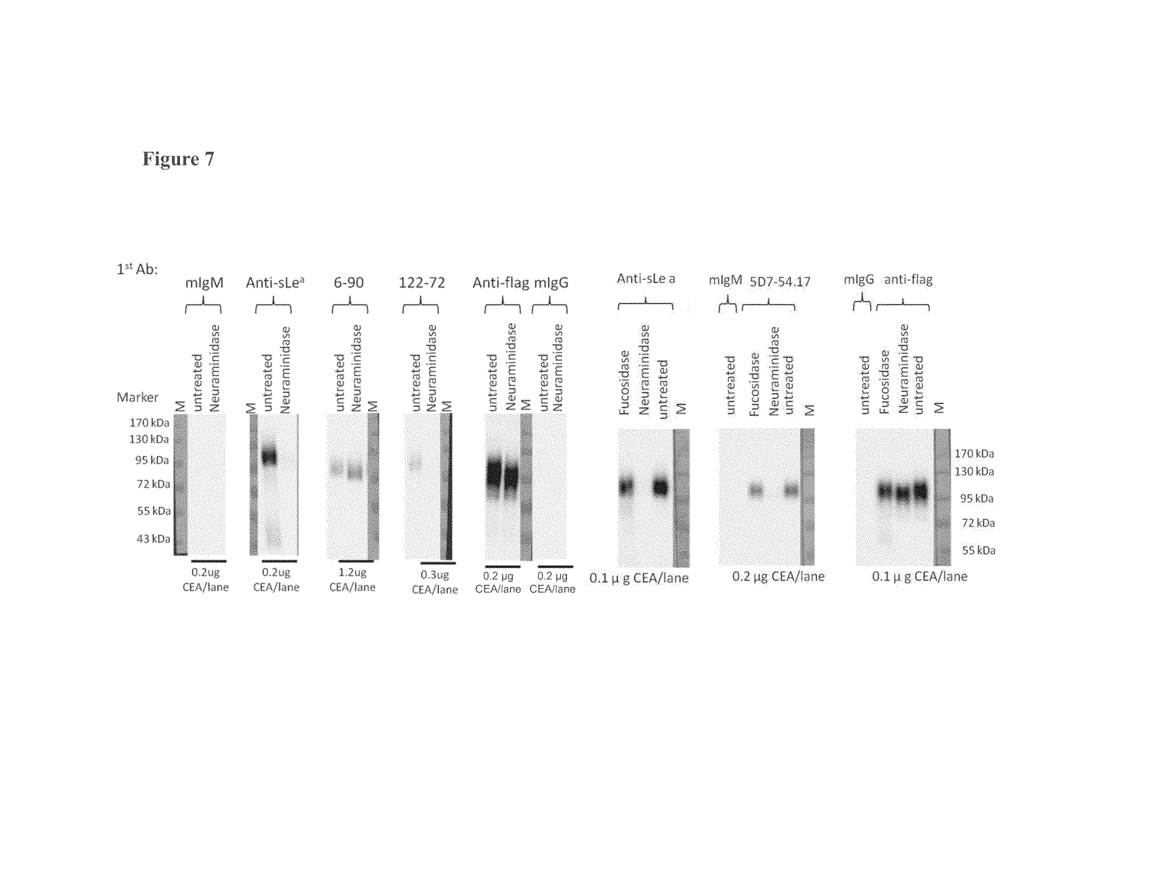

To determine whether carbohydrate modifications on transferrin receptors were involved in the binding of 6-90, 122-72 and 5D7-54.17 antibodies to cancer cells, a recombinant FLAG-tagged human CEA (rCEA) fragment was expressed from Colo205 cells, which was then used to identify glyco-epitopes of these antibodies. Western blot confirmed the presence of antibody epitopes for 6-90, 122-72 and 5D7-54.17 in this recombinant fragment (FIG. 7, untreated). After glycosidase treatment, binding of these antibodies to rCEA protein was evaluated by western blot. In addition, an anti-flag antibody was used to demonstrate equal loading amount and protein integrity after enzyme treatment. Mouse IgM was used as isotype control. Anti-sialyl lewis^{a} antibody (clone name KM231, EMD Chemicals, Inc. Cat. No. 565942) was used to demonstrate effective enzyme function of α2-3,6,8-Neuraminidase.

A clear reduction in binding of 122-72 and 5D7-54.17 to rCEA was observed after protein treated with α2-3,6,8-Neuraminidase (FIG. 7), indicating that the epitopes for 122-72 and 5D7-54.17 contain sialyl-moiety. Recognition by the 6-90 antibody was not affected after neuraminidase treatment, indicating that the epitope for 6-90 does not contain sialyl-moiety.

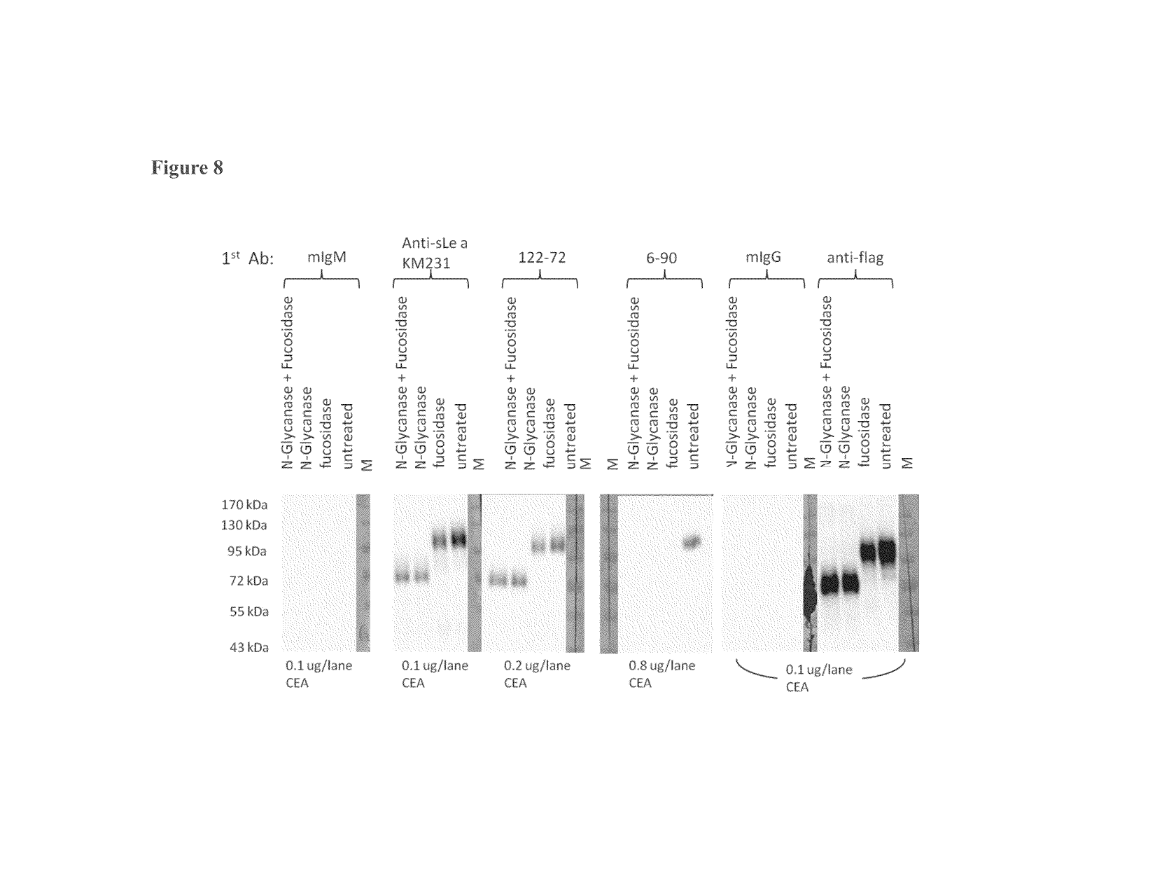

After treatment with α-1→(2,3,4)-fucosidase, N-glycanase, or a combination of both, the binding of 6-90 to rCEA was totally ablated (FIG. 8), indicating that the epitope for 6-90 contains fucose-moiety. Since the epitopes for 122-72 and 5D7-54.17 contain sialyl-moiety, which likely prevents digestion of adjacent fucose by fucosidase, the possible contribution of fucose-moiety in the epitopes for 122-72 and 5D7-54.17 could not be tested by this assay.

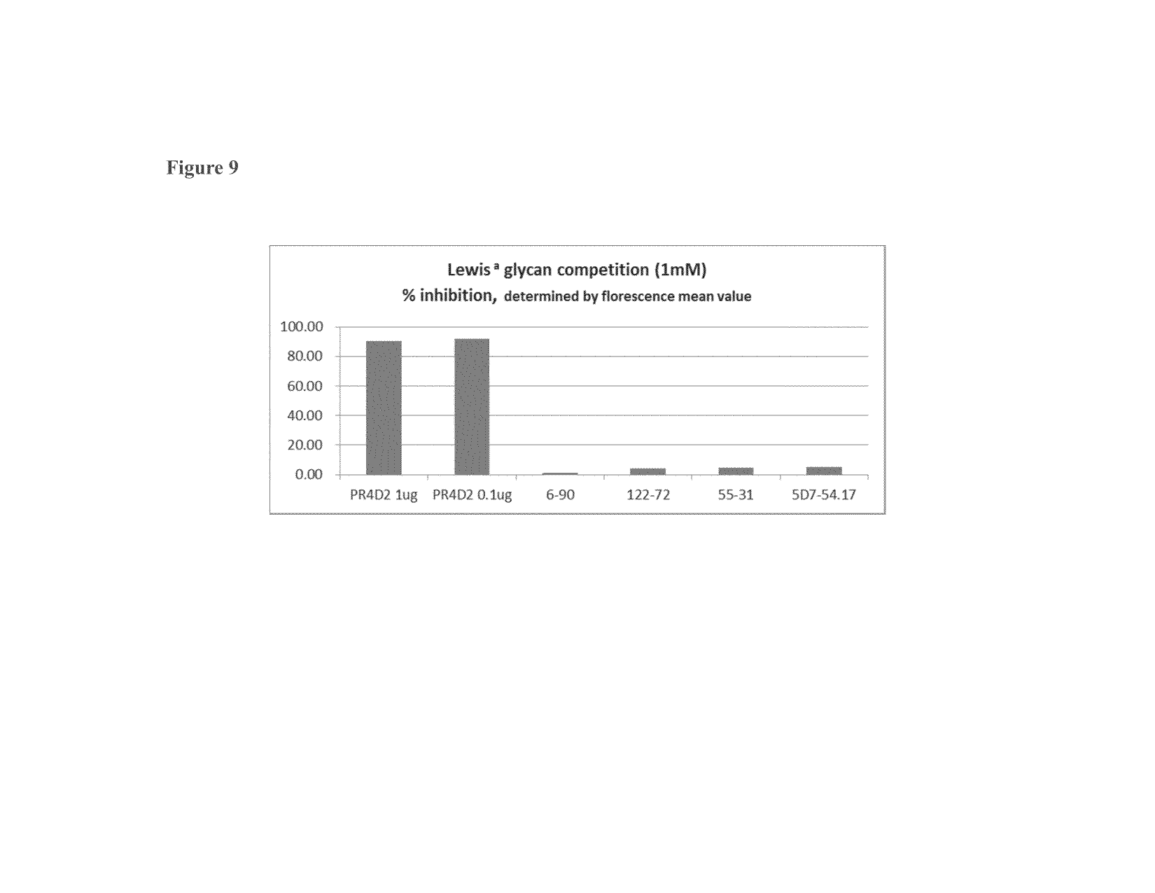

Glycan competition assay was performed to examine whether trisaccharide lewis a can interfere with the binding of 6-90, 122-72, 55-31 and 5D7-54.17 to Panc 02.03B cells. Anti-Lewis^{a} antibody (clone PR4D2) was used as a positive control. The results in FIG. 9 show that none of these antibodies can be competed off by lewis a in binding to Panc 02.03B.

### Example 6

### Effects of Anti-TfR Antibody Based Antibody Drug Conjugate (ADC) in Inhibiting Tumor Growth

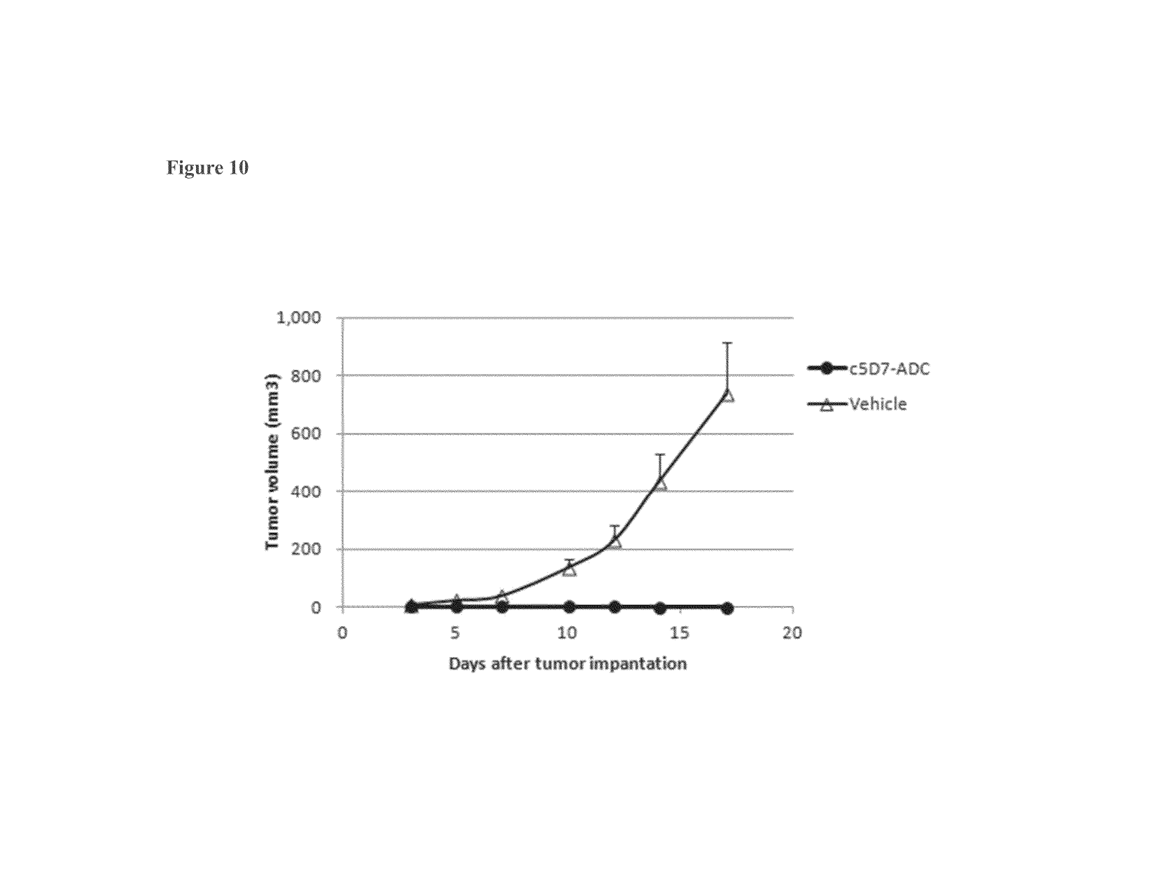

The chimeric 5D7-54.17 antibody (c5D7) was used in preparing an antibody drug conjugate (ADC), c5D7-monomethyl dolastatin 10 ADC (see below for the methods of making the ADC). The anti-tumor activity of c5D7-monomethyl dolastatin 10 ADC was evaluated in vivo on DLD-1 transplanted SCID mice. Treatment was initiated at days 1 and 5 following tumor inoculation with 3 mg/kg of ADC or vehicle. Compared to the vehicle group in which tumor approached 500 mm³ at day 14, c5D7-ADC completely suppressed tumor growth throughout the study period (FIG. 10). Body weight of mice from either group remained unchanged after treatment (25 g on average). The data shows that cancer targeting delivery of cytotoxic drug by the anti-transferrin receptor c5D7 was able to effectively inhibit tumor growth in vivo.

### Detailed Description for Materials and Methods Used in the Examples Above.

### Generation of Transferrin Receptor Antibodies

Immunization with cancer cells membrane extracts/purified transferrin receptor: Balb/c mice were immunized and boosted three times with 300 μg of pancreatic cancer cell line Panc 02.03B membrane fraction or 50 ng of transferrin receptor purified from Panc 02.03B, and spleen cells were fused with P3X63 myeloma cells. Hybridomas were cultured and selected with DMEM supplemented with 10% FBS (Hyclone®) and HAT (Hybri-Max®, Sigma H0262, at a final concentration of 100 μM hypoxanthine, 0.4 μM aminopterin, 16 μM thymidine). Three hybridoma clones, 6-90 (IgM, κ), 55-31 (IgM, κ) and 122-72 (IgM, κ), were selected due to their ability to recognize cancer-specific modification of human transferrin receptor on Panc 02.03B cells.

Balb/c mice were immunized and boosted once with 300 μg of membrane fraction of lung cancer cell line H358, and spleen cells were fused with P3X63 myeloma cells. Hybridomas were cultured in the medium as described above, and screened for positive H358 binding by FACS. One hybridoma clone 5D7-54.17, which was identified as mouse IgM, κ, was further selected due to its ability to recognize cancer-specific modification of human transferrin receptor on lung cancer cell line H358.

Cellular ELISA: 96-well flat-bottom plate was seeded with 6˜7×10⁴ Panc 02.03B cells per well in 100 μl complete medium and cultured at 37° C. overnight for complete attachment of cells to the wells. Next day, culture medium was discarded and 50 μl of hybridoma culture medium was added into 96-well flat-bottom for 1 hour at 4° C. After washing with PBS, pH7.2, the Panc 02.03B cells were fixed by 2% Paraformaldehyde for 20 minutes at room temperature. After one washing with PBS, pH7.2, 50 μl of 1:5000 diluted HRP-conjugated goat anti-mouse Ig (Southern biotech™, Cat. No. 1010-05) was added and incubated for 0.5-1 hour at 4° C. After one washing with PBS (pH7.2), 50 μl of substrate containing TMB (BD™, Cat. No. 555214) was added and incubated for 5-10 minutes till color development. The reaction was stopped by the addition of 50 μl 0.5N H₂SO₄ solution and the color development was measured at OD₄₅₀ by Molecular Devices VERSEmax reader.

### Cloning of the CDR of Light and Heavy Chains of Antibodies

The cDNAs of the antibody light and heavy chain variable regions were amplified by PCR, and the synthesized cDNAs were subcloned into pCRII (Invitrogen™) for sequence determination. Nucleotide sequences were obtained from several independent clones and analyzed. cDNA sequences from several independent clones with identical sequences were selected and further verified by FACS to encode the light or heavy chain variable region of each antibody.

### Detection of Transferrin Receptor by Immunoprecipitation and Western Blot

2×10⁷ H358 cells or 3.5×10⁷ activated T were lysed with lysis buffer (TN buffer, 50 mM Tris-HCl, 150 mM NaCl, 1% NP-40 and protease inhibitor (0.5% Deoxycholate was added in addition when preparing activated T cell lysate)), and immunoprecipitated using anti-transferrin receptor antibody MEM-189 (Abcam™, Cat. No. ab1086). The precipitated proteins were separated by 8% SDS-PAGE and transferred onto NC membrane followed by blocking with 7% defatted milk at room temperature, then blotted with antibodies 6-90, 55-31, 122-72, 5D7-54.17 or anti-transferrin receptor polyclonal antibody C20 (as positive control, Santa Cruz Biotechnology™, Cat. No. Sc-7087) at room temperature for 2-18 hours. After washing with TBS-T (20 mM Tris-base, pH 7.4, 150 mM NaCl, 0.125% Tween 20), membrane was incubated with horseradish peroxidase-conjugated goat anti-mouse IgG(H+L) (Southern biotech™, Cat. No. 1031-05) or rabbit anti-goat IgG (Santa Cruz Biotechnology™, Cat. No. Sc-2768) at room temperature for 1 hour. After washing with TBS-T, the protein was visualized using ECL detection system (Fujifilm LAS-4000).

### Characterization of Anti-Transferrin Receptor (TfR) Clones

Antibodies preparation: Culture supernatant of hybridoma clones 6-90, 55-31, 122-72 and 5D7-54.17 used in the current studies was produced at AbGenomics B.V. Taiwan Branch, Taipei, Taiwan. The isotype control, mouse IgM kappa, was purchased from BD Pharmingen™ (Cat. No. 557275). Goat-anti-mouse IgM-PE was from Southern Biotech™ (Cat. No. 1022-09).

The normal cell preparation methods are described below in detail.

Preparation of PBMC and PMN: The whole blood was diluted with an equal volume of PBS and mixed with 1/10 volume of 15% Dextran solution. After sitting at room temperature for 20 min, the upper layer, rich in WBC, was washed and resuspended in PBS, and half volume of Ficoll (Ficoll-Paque™ PLUS, GE Healthcare; Cat. No. 17-1440-03) was added to the bottom of tube. After centrifugation at 2200 rpm for 15 min (rotor: SORVALL, PN11788 and centrifuge: SORVALL, RT7), PBMC concentrated in the interface, were collected and washed with large volumes of FACS buffer (2% FBS in PBS) for future use. The PMN-rich pellet was collected, from which residual RBC was removed by hypotonic lysis in 0.2% NaCl.

Preparation of Red blood cells and platelets: 9 ml of whole blood was mixed with 1 ml of sodium citrate buffer, pH6.5, and centrifuged at 200×g for 15 min at room temperature. After centrifugation, the cellular (lower) layer was used to prepare RBC as described below. The plasma (upper, platelet-rich) layer was carefully transferred into a clean centrifuge tube, and mixed with 1/10 volume of ACD buffer (85 mM sodium citrate, 64.54 mM citric acid, 75.5 mM D-glucose). After centrifugation at 900×g for 5 min at room temperature, plasma was discarded and the platelet pellet was resuspended in 1 ml of HEPES-Tyrode buffer (134 mM NaCl, 2.9 mM KCl, 5 mM glucose, 12 mM NaHCO₃, 0.34 mM NaH₂PO₄, 5 mM HEPES, 1% BSA, pH 7.4). The OD₆₀₀ of the resulting platelet solution was measured. The platelet solution was first adjusted to OD₆₀₀ 0.25˜0.3 and then diluted 5 times for binding test. The cellular (lower) phase was mixed with an equal volume of PBS and carefully layered on top of Ficoll (Ficoll-Paque™ PLUS, GE Healthcare, Cat. No. 17-1440-03). After centrifugation at 2400 rpm for 15 min (rotor: SORVALL, PN11788 and centrifuge: SORVALL, RT7), the RBC pellet was resuspended in 10 times volume of PBS and counted. Aliquots of 1×10⁶ cells were used in antibody binding tests.

Preparation of activated T cells: Human PBMC were activated by PHA (5 ug/ml, Roche Diagnostics GmbH), expanded in IL-2 (5 ng/ml, R&D System) containing media until day 4˜day 5 for binding assay or immunoprecipitation/western blot analysis.

### Preparation of Cancer Cell Lines

Human cancer cell lines H358 (Cat. No. CRL-5807), H727 (Cat. No. CRL-5815), SU.86.86 (Cat. No. CRL-1837), SK-OV-3 (Cat. No. HTB-77), PC3 (Cat. No. CRL-1435), KLE (Cat. No. CRL-1622) and DU 145 (Cat. No. HTB-81) were obtained from the American Type Culture Collection (ATCC), Manassas, Va., USA.

Human cancer cell lines A549 (Cat. No. BCRC 60074), H520 (Cat. No. BCRC 60124), PANC-1 (Cat. No. BCRC 60284), SNU-16 (Cat. No. BCRC 60212), NCI-N87 (Cat. No. BCRC 60217), KATO III (Cat. No. BCRC 60200), COLO 205 (Cat. No. BCRC 60054), DLD-1 (Cat. No. BCRC 60132), WiDr (Cat. No. BCRC 60157), Hs578T (Cat. No. BCRC 60120), T47D (Cat. No. BCRC 60250), MDA-MB-453 (Cat. No. BCRC 60429), 22Rv1 (Cat. No. BCRC 60545), Hep 3B2.1-7 (Cat. No. HB-8064), Hep G2 (Cat. No. BCRC 60025), HEC-1-A (Cat. No. BCRC 60552) and PLC/PRF/5 (Cat. No. BCRC 60223) were obtained from Food Industry Research and Development Institute, Hsin-chu, Taiwan. Human cancer cell line OMC-3 (Cat. No. RCB0755) was obtained from Riken BioResource Center, Ibaraki, Japan.

Cells were grown in the medium listed below, and cultured at 37° C. in a humidified atmosphere with 5% CO₂.

TABLE 5
Culture conditions for various cancer cell lines

| | |
|---|---|
| H358, H520, | RPMI Medium 1640 (GIBCO ™, Cat. No. 22400) |
| H727, SU.86.86, | supplemented with 10% FBS (GIBCO ™, Cat. No. |
| SNU-16, NCI-N87, | 26140) and 100 U/mL penicillin/100 ug/mL |
| COLO 205, PC3 | streptomycin (GIBCO ™, Cat. No. 15140). |
| DLD-1, 22Rvl | RPMI Medium 1640 (GIBCO ™, Cat. No. 22400) |
| | supplemented with 10% FBS, 1 mM sodium |
| | pyruvate (GIBCO ™, Cat. No. 11360), and |
| | 100 U/mL penicillin/100 ug/mL streptomycin |
| | (GIBCO ™, Cat. No. 15140). |
| T47D | RPMI Medium 1640 (GIBCO ™, Cat. No. 22400) |
| | supplemented with 10% FBS, 10 ug/mL bovine |
| | insulin (Sigma ™, Cat. No. SI-I6634), and |
| | 100 U/mL penicillin/100 μg/mL streptomycin |
| | (GIBCO ™, Cat. No. 15140). |
| WiDr, DU 145, | Minimum essential medium Eagle (GIBCO ™, |
| Hep 3B2.1-7, | Cat. No. 11095) supplemented with 10% FBS, |
| Hep G2, | 0.1 mM non-essential amino acids (GIBCO ™, |
| PLC/PRF/5 | Cat. No. 11140-050), 1 mM sodium pyruvate |
| | (GIBCO ™, Cat. No. 11360), and 100 U/mL |
| | penicillin/100 ug/mL streptomycin (GIBCO ™, |
| | Cat. No. 15140). |
| PANC-1 | Dulbecco's modified Eagle's medium |
| | (GIBCO ™, Cat. No. 11965) supplemented |
| | with 10% FBS and 100 U/mL penicillin/100 |
| | ug/mL streptomycin (GIBCO ™, Cat. No. 15140). |
| Hs578T | Dulbecco's modified Eagle's medium |
| | (GIBCO ™, Cat. No. 11965) supplemented |
| | with 10% FBS, 10 ug/mL bovine insulin |
| | (Sigma ™, Cat. No. SI-I6634), and 100 |
| | U/mL penicillin/100 ug/mL streptomycin |
| | (GIBCO ™, Cat. No. 15140). |
| A549 | Ham's F12K medium (GIBCO ™, Cat. No. |
| | 21127) supplemented with 10% FBS and 100 U/mL |
| | penicillin/100 ug/mL streptomycin |
| | (GIBCO ™, Cat. No. 15140). |
| OMC-3 | Ham's F12 medium (GIBCO ™, Cat. No. 11765) |
| | supplemented with 10% NBS (Newborn Calf |
| | Serum) (Invitrogen ™, Cat. No. 16010-159) |
| | and 100 U/mL penicillin/100 ug/mL streptomycin |
| | (GIBCO ™, Cat. No. 15140). |
| SK-OV-3, | McCoy's 5a Medium Modified (GIBCO ™, Cat. |
| HEC-1-A | No. 16600) supplemented with 10% FBS and |
| | 100 U/mL penicillin/100 ug/mL streptomycin |
| | (GIBCO ™, Cat. No. 15140). |
| KEL | Mixed Dulbecco's modified Eagle's medium |
| | (GIBCO ™, Cat. No. 11965):F12 Medium |
| | (GIBCO ™, Cat. No. 11765) (50%:50%) |
| | supplemented with 10% FBS and 100 U/mL |
| | penicillin/100 ug/mL streptomycin (GIBCO ™, |
| | Cat. No. 15140). |

The pancreatic cancer cell line Panc 02.03B was adapted from Panc 02.03 (originally obtained from ATCC Cat. No. CRL-2553), and cultured in RPMI Medium 1640 (GIBCO™, Cat. No. 22400) supplemented with 15% FBS, 1 mM sodium pyruvate (GIBCO™, Cat. No. 11360), and 100 U/ml penicillin/100 ug/ml streptomycin (GIBCO™, Cat. No. 15140).

The breast cancer cell line MDA-MB-453 was grown in Leibovitz's L-15 medium (GIBCO™, Cat. No. 11415) supplemented with 10% FBS, 2 mM L-glutamine and 100 U/ml penicillin/100 ug/ml streptomycin (GIBCO™, Cat. No. 15140), and cultured at 37° C. in a humidified atmosphere without CO₂.

### Binding of Anti-Transferrin Receptor Antibodies to N-Flag TfR CHO Cell, Activated T Cells, RBC, PMN, Monocyte, Lymphocytes and Platelet

1×10⁵ cells were seeded in each well of a v-bottomed 96-well plate and incubated with 50 μl of hybridoma culture supernatant at 1× and 10× dilution, or isotype control antibody mouse IgM, κ (BD™, Cat. No. 557275) at concentration of 1 μg/ml. A 300× dilution of human cell hyper-immune serum was used as binding positive control for all cell types. PE-conjugated anti-human CD41a (Southern Biotech™, Cat. No. 9391-09) was used as a positive control for staining human platelets. Anti-sialyl Lewis x antibody (Millipore™, Cat. No. MAB2096) was used as a positive control for staining human PMN. Anti-human CD31 (Hycult™, Cat. No. HM2039) was used as a positive control for staining HUVEC. After 30 minutes incubation at 4° C., cells were washed twice with 200 μl FACS buffer (1×PBS containing 1% FBS), stained with 50 μl of 1 μg/ml goat F(ab′)2-anti-mouse IgG(H+L)-PE (Southern Biotech™, Cat. No. 1032-09) or 1 μg/ml goat F(ab′)2-anti-mouse IgM(H+L)-PE (Southern Biotech™, Cat. No. 1022-09) in FACS buffer. After 30 minutes of incubation at 4° C., cells were washed twice with FACS buffer and analyzed by flow cytometry (BD LSR, BD Life Sciences).

### Binding of Anti-Transferrin Receptor Antibodies to Cancer Cell Lines

1×10⁵ cells were seeded per well in a v-bottomed 96-well plate and incubated with 50 μl of hybridoma culture supernatant (at 1× and 5× dilution) or isotype control antibody mouse IgM, κ (BD™, Cat. No. 557275) (at concentration of 5 μg/ml). Mouse hyper-immune serum (HPS 300× dilution) and anti-transferrin receptor antibody MEM-189 were used as binding positive controls for all cell types. After 30 minutes of incubation at 4° C., cells were washed twice with 200 μl FACS buffer (1×PBS containing 1% FBS), stained with 50 μl of 1 μg/ml goat F(ab′)2-anti-mouse IgM(H+L)-PE (Southern Biotech™, Cat. No. 1022-09) in FACS buffer and then incubated at 4° C. for 30 minutes. Cells were washed twice with FACS buffer and analyzed by flow cytometer (BD LSR, BD Life Sciences).

### Induction of Apoptosis by Anti-Transferrin Receptor Antibodies

1×10⁵ of tested cancer cells were seeded into the wells of 96-well plates. Hybridoma culture medium and control antibodies (at indicated dilutions or concentrations) were prepared freshly in culture medium and added to each well. The treated cells were kept at 37° C. incubator for overnight before FACS analysis for apoptosis. For cellular apoptosis assay, Annexin V staining was measured using Annexin-V-FITC Apoptosis Detection Kit (Strong Biotech™, Cat. No. AVK250) following the manufacturer's instruction. Briefly, cells were incubated with Annexin V binding buffer containing 1 μl Annexin V-FITC at room temperature for 15 minutes in the dark, followed by 2 times of wash with 200 μl of Annexin V binding buffer. 1 μl of propidium iodide (PI) was added before FACS analysis. All flow cytometric analyses were performed on a BD-LSR flow cytometer (Becton Dickinson) using the Cell Quest software. The Annexin V positive and/or PI positive cells were considered apoptotic cells.

### Internalization Assay

1×10⁵ cells were cultured on coverslips for 3 days prior to staining. On the day of assay, cells were treated with 150 μl of tested antibodies at 4° C. (negative control) or 37° C. (to allow internalization) for 4 hours. After removing the unbound antibodies by cold PBS wash, cells were fixed with 150 μl of 3.7% formaldehyde at 4° C. for 20 min Cells were washed again with cold PBS, and permeabilized with 150 μl of 3.7% formaldehyde containing 0.5% of Triton X-100 at 4° C. for 20 minutes. After one wash with cold PBS, the secondary antibody FITC-conjugated Goat F(ab)₂ Anti-Mouse IgM (1 μg/ml) (Southern Biotech™, Cat. No. 1022-02) was added and incubated at room temperature for 20 minutes. Finally, cells were washed once with cold PBS and examined for immunofluorescence by confocal laser microscopy (LSM700-CarlZeiss).

### Determination of Antibody Epitope

Glycosidase digestion: Recombinant CEA (rCEA) fragment, expressed and purified from Colo205 cells (which was shown to contain epitopes for some of the hybridoma clones), was used to identify glycol-epitopes of anti-transferrin receptor antibodies. The rCEA protein (0.2˜1.2 μg) was incubated with the following glycosidases for 18 hours at 37° C. in 50˜75 μl of the buffer recommended by the manufacturers: α2-3,6,8-Neuraminidase, (EMD™, Cat. No. 480717), α-1→(2,3,4)-Fucosidase (Sigma-Aldrich™, Cat. No. f1924-1VL), Glyko® N-Glycanase™ (ProZyme™, Cat. No. ws0041). The treated protein samples were subsequently analyzed by SDS-PAGE and blotted with the tested antibodies.

Competition Assay with Oligosaccharides

Panc 02.03B cells were seeded at 1×10⁵ cells per well in a v-bottomed 96-well plate. Cells were incubated with 300-900 fold diluted hybridoma culture supernatant, in the absence or presence of 1 mM oligosaccharide competitor Lewis a (Calbiochem® CB-434626) at a final volume of 50 μl. After 1 hour incubation at 4° C., cells were washed twice with 200 μl FACS buffer (1×PBS containing 1% FBS), stained with 50 μl of 1 μg/ml goat F(ab′)2-anti-mouse IgG(H+L)-PE (Southern Biotech, Cat. No. 1032-09) or 1 μg/ml goat F(ab′)2-anti-mouse IgM(H+L)-PE (Southern Biotech™, Cat. No. 1022-09) in FACS buffer, and incubated at 4° C. for 30 minutes. Cells were then washed twice with FACS buffer and analyzed by flow cytometry (BD LSR, BD Life Sciences). Anti-Lewis^{a} antibody (clone name PR4D2, Chemicon™, Cat. No. MAB438) was used as positive control in the assay.

The competition ability was expressed as percent reduction, calculated by 100%×[(MFI in the absence of oligosaccharide competitor)−(MFI in the presence of each ligosaccharide competitor)]/(MFI in the absence of oligosaccharide competitor).

### Preparation of Antibody Drug Conjugates (ADCs)

Chimeric 5D7-54.17 (c5D7) was produced from Flp-In CHO cells transfected with expression vector, pcDNA5-FRT-hIgG1, containing the heavy and light chain variable region genes of murine 5D7-54.17. Table 6 below shows the amino acid sequences of the heavy chain sequence and light chain sequence of c5D7 antibody.

TABLE 6(A)
c5D7 Heavy chain sequence (SEQ ID NO: 17) (Kabat CDRs in some embodiments
are underlined; the sequence in constant region is italicized)

(SEQ ID NO: 17)

| | |
|---|---|
| 1 | EVQLQQSGPEVVKPGASMKMSCKTSGYKFTGYYMDWVKQSLGASFEWIGRVIPSNGDTRY |
| 61 | NQKFEGKATLTVDRSSSTAYMELNSLTSEDSAVYYCARKPLSGNAADYWGQGTSVTVSTA |
| 121 | STKGPSVFPLAPSSKSTSGGTAALGCLVKDYFPEPVTVSWNSGALTSGVHTFPAVLQSSG |
| 181 | LYSLSSVVTVPSSSLGTQTYICNVNHKPSNTKVDKKVEPKSCDKTHTCPPCPAPELLGGP |
| 241 | SVFLFPPKPKDTLMISRTPEVTCVVVDVSHEDPEVKFNWYVDGVEVHNAKTKPREEQYNS |
| 301 | TYRVVSVLTVLHQDWLNGKEYKCKVSNKALPAPIEKTISKAKGQPREPQVYTLPPSRDEL |
| 361 | TKNQVSLTCLVKGFYPSDIAVEWESNGQPENNYKTTPPVLDSDGSFFLYSKLTVDKSRWQ |
| 421 | QGNVFSCSVMHEALHNHYTQKSLSLSPGK |

TABLE 6(B)
c5D7 Light chain sequence (SEQ ID NO: 18) (Kabat CDRs in some embodiments
are underlined; the sequence in constant region is italicized)

(SEQ ID NO: 18)

| | |
|---|---|
| 1 | ETTVTQSPASLSVATGEKVTIRCITSTDIDDDMNWYQQKPGEPPKLLISDGNTLRPGVPS |
| 61 | RFSSSGYGTDFVFTIENTLSEDITDYYCMQSDNMPFTFGSGTKLEIKRTVAAPSVFIFPP |
| 121 | SDEQLKSGTASVVCLLNNFYPREAKVQWKVDNALQSGNSQESVTEQDSKDSTYSLSSTLT |
| 181 | LSKADYEKHKVYACEVTHQGLSSPVTKSFNRGEC |

The c5D7 antibody was conjugated to the cytotoxic drug monomethyl dolastatin 10 to evaluate its anti-tumor effect in vivo via a piperazin containing linker (see structure below). The ADCs may be made using methods described in U.S. Provisional Application No. 61/745,448 filed Dec. 21, 2012, the contents of which are incorporated by reference herein in their entirety. In one example, purified c5D7 was firstly reduced with 3.0 equivalents of TCEP (or tris(2-carboxyethyl)phosphine) in 0.025 M sodium borate pH 8, 0.025 M NaCl, 1 mM DTPA (or Pentetic acid or diethylene triamine pentaacetic acid) for 2 h at 37° C. The protein concentration was quantified using an absorbance value of 1.346 at 280 nm for a 1.0 mg/mL solution, and the molar concentration determined using a molecular weight of 145,194 g/mol. The concentration of mAb-cysteine thiols produced was determined by titrating with DTNB (or 5,5′-dithiobis-(2-nitrobenzoic acid)). Typically 4.0 to 4.5 thiols/mAb was produced when 3.0 molar equivalents of TCEP were used. Partially reduced c5D7 was alkylated with 2.4 molar of maleimidocaproyl-monomethyl dolastatin 10/mAb-cysteine thiol. The alkylation reaction was performed at 10° C. for 30 min. Cysteine (1 mM final) was used to quench any unreacted, excess maleimidocaproyl-monomethyl dolastatin 10 drug. The resultant ADCs were changed to phosphate buffered saline by dialysis overnight at 4° C.

Structure for the Linker-Drug portion of the Antibody-Drug conjugate:

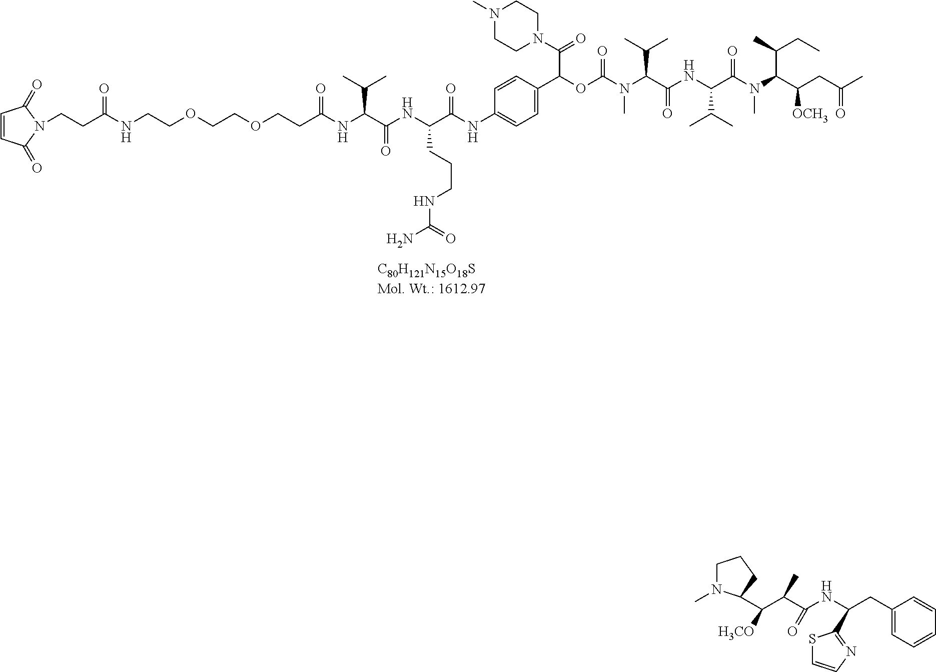

### ADC Treatment in Cancer Xenograft Model

To establish a subcutaneous xenograft model, 5×10⁶ DLD-1 cells were implanted into the right flank of C.B-17 SCID mice (Lasco, Taipei, Taiwan). Drug-conjugated c5D7 ADC was administered intravenously at 3 mg/kg at days 1 and 5 post tumor inoculation. Tumor volume was measured twice weekly with a caliper in two perpendicular dimensions, and calculated according to the formula 0.52×length×width×width.

Although the foregoing invention has been described in some detail by way of illustration and example for purposes of clarity of understanding, the descriptions and examples should not be construed as limiting the scope of the invention.

What is claimed is:

1. An isolated antibody that specifically binds to a carbohydrate on a transferrin receptor expressed by nonhematopoietic cancer cells but does not specifically bind to a transferrin receptor expressed by activated T cells or by Jurkat cells, wherein the binding of the antibody to the transferrin receptor is not inhibited by a carbohydrate comprising a Le structure.

2. The antibody of claim 1, wherein the antibody is a monoclonal antibody.

3. The antibody of claim 2, wherein the antibody binds to an epitope comprising a fucose moiety.

4. The antibody of claim 2, wherein the antibody binds to an epitope comprising a sialyl moiety.

5. The antibody of claim 2, wherein the antibody binds to an epitope not comprising a sialyl moiety.

6. The antibody of claim 1, wherein the binding of the antibody to the transferrin receptor is not inhibited by a carbohydrate comprising a Le^{b}, Le^{y}, or Le^{x} structure.

7. The antibody of claim 1, wherein the antibody competes for binding to the transferrin receptor with an antibody comprising a heavy chain variable region comprising the three complementarity determining regions (“CDRs”) from SEQ ID NO:1 and/or a light chain variable region comprising the three CDRs from SEQ ID NO:3.

8. The antibody of claim 1, wherein the antibody comprises a heavy chain variable region comprising the three CDRs from SEQ ID NO:1 and/or a light chain variable region comprising the three CDRs from SEQ ID NO:3.

9. The antibody of claim 1, wherein the antibody comprises (i) a heavy chain variable region comprising a sequence at least about 95% identical to amino acids 20-138 of SEQ ID NO:1 and/or (ii) a light chain variable region comprising a sequence at least about 95% identical to amino acids 20-132 of SEQ ID NO:3.

10. The antibody of claim 9, wherein the antibody comprises a heavy chain variable region comprising amino acids 20-138 of SEQ ID NO:1 and/or a light chain variable region comprising amino acids 20-132 of SEQ ID NO:3.

11. The antibody of claim 1, wherein the antibody competes for binding to the transferrin receptor with an antibody comprising a heavy chain variable region comprising the three CDRs from SEQ ID NO:5 and/or a light chain variable region comprising the three CDRs from SEQ ID NO:7.

12. The antibody of claim 1, wherein the antibody comprises a heavy chain variable region comprising the three CDRs from SEQ ID NO:5 and/or a light chain variable region comprising the three CDRs from SEQ ID NO:7.

13. The antibody of claim 1, wherein the antibody comprises (i) a heavy chain variable region comprising a sequence at least about 95% identical to amino acids 20-138 of SEQ ID NO:5 and/or (ii) a light chain variable region comprising a sequence at least about 95% identical to amino acids 21-128 of SEQ ID NO:7.

14. The antibody of claim 13, wherein the antibody comprises a heavy chain variable region comprising amino acids 20-138 of SEQ ID NO:5 and/or a light chain variable region comprising amino acids 21-128 of SEQ ID NO:7.

15. The antibody of claim 1, wherein the antibody competes for binding to the transferrin receptor with an antibody comprising a heavy chain variable region comprising the three CDRs from SEQ ID NO:9 and/or a light chain variable region comprising the three CDRs from SEQ ID NO:11.

16. The antibody of claim 1, wherein the antibody comprises a heavy chain variable region comprising the three CDRs from SEQ ID NO:9 and/or a light chain variable region comprising the three CDRs from SEQ ID NO:11.

17. The antibody of claim 1, wherein the antibody comprises (i) a heavy chain variable region comprising a sequence at least about 95% identical to amino acids 20-136 of SEQ ID NO:9 and/or (ii) a light chain variable region comprising a sequence at least about 95% identical to amino acids 21-134 of SEQ ID NO:11.

18. The antibody of claim 17, wherein the antibody comprises a heavy chain variable region comprising amino acids 20-136 of SEQ ID NO:9 and/or a light chain variable region comprising amino acids 21-134 of SEQ ID NO:11.

19. The antibody of claim 1, wherein the antibody competes for binding to the transferrin receptor with an antibody comprising a heavy chain variable region comprising the three CDRs from SEQ ID NO:13 and/or a light chain variable region comprising the three CDRs from SEQ ID NO:15.

20. The antibody of claim 1, wherein the antibody comprises a heavy chain variable region comprising the three CDRs from SEQ ID NO:13 and/or a light chain variable region comprising the three CDRs from SEQ ID NO:15.

21. The antibody of claim 1, wherein the antibody comprises (i) a heavy chain variable region comprising a sequence at least about 95% identical to amino acids 20-138 of SEQ ID NO:13 and/or (ii) a light chain variable region comprising a sequence at least about 95% identical to amino acids 23-130 of SEQ ID NO:15.

22. The antibody of claim 21, wherein the antibody comprises a heavy chain variable region comprising amino acids 20-138 of SEQ ID NO:13 and/or a light chain variable region comprising amino acids 23-130 of SEQ ID NO:15.

23. The antibody of claim 1, wherein the antibody competes for binding to the transferrin receptor with an antibody comprising a heavy chain variable region comprising the three CDRs from SEQ ID NO:17 and/or a light chain variable region comprising the three CDRs from SEQ ID NO:18.

24. The antibody of claim 1, wherein the antibody comprises a heavy chain variable region comprising the three CDRs from SEQ ID NO:17 and/or a light chain variable region comprising the three CDRs from SEQ ID NO:18.

25. The antibody of claim 1, wherein the antibody comprises (i) a heavy chain variable region comprising a sequence at least about 95% identical to amino acids 1-119 of SEQ ID NO:17 and/or (ii) a light chain variable region comprising a sequence at least about 95% identical to amino acids 1-108 of SEQ ID NO:18.

26. The antibody of claim 25, wherein the antibody comprises a heavy chain variable region comprising amino acids 1-119 of SEQ ID NO:17 and/or a light chain variable region comprising amino acids 1-108 of SEQ ID NO:18.

27. The antibody of claim 1, wherein the antibody is a humanized antibody.

28. The antibody of claim 1, wherein the antibody is a chimeric antibody.

29. The antibody of claim 1, wherein the antibody is a human antibody.

30. The antibody of claim 1, wherein the nonhematopoietic cancer cells are pancreatic cancer cells, gastric cancer cells, colorectal cancer cells, lung cancer cells, ovarian cancer cells, endometrial cancer cells, prostate cancer cells, breast cancer cells, or liver cancer cells.

31. The antibody of claim 1, wherein the antibody does not bind to a transferrin receptor expressed by CHO cells, red blood cells, platelets, HUVEC cells, monocytes, PMN, or T cells.

32. The antibody of claim 1, wherein the antibody is internalized after binding to the transferrin receptor on cell surface of the cancer cells.

33. The antibody of claim 1, wherein the antibody is capable of inducing apoptosis of the cancer cells after binding to the transferrin receptor on cell surface of the cancer cells in the absence of cytotoxin conjugation and immune effector function.

34. The antibody of claim 1, wherein the antibody is conjugated to a cytotoxin.

35. The antibody of claim 1, wherein the antibody is conjugated to a label.

36. A pharmaceutical composition comprising the antibody of claim 1 and a pharmaceutically acceptable carrier.

37. A polynucleotide comprising a nucleic acid sequence encoding the antibody of claim 1.

38. A vector comprising a nucleic acid sequence encoding the antibody of claim 1.

39. A host cell comprising the vector of claim 38.

40. A method of producing an antibody comprising culturing the host cell of claim 39 that produces the antibody and recovering the antibody produced by the host cell.

41. A method of treating nonhematopoietic cancer in an individual comprising administering to the individual an effective amount of an antibody of claim 1.

42. The method of claim 41, wherein the nonhematopoietic cancer is pancreatic cancer, gastric cancer, colorectal cancer, lung cancer, ovarian cancer, prostate cancer, endometrial cancer, breast cancer, or liver cancer.

43. The method of claim 41, wherein the antibody is conjugated to a cytotoxin.

44. A method of treating nonhematopoietic cancer in an individual comprising administering to the individual an amount of an antibody of claim 1 and an amount of another anti-cancer agent, whereby the antibody and the anti-cancer agent in conjunction provide effective treatment of cancer in the individual.

45. The method of claim 44, wherein the nonhematopoietic cancer is pancreatic cancer, gastric cancer, colorectal cancer, lung cancer, ovarian cancer, prostate cancer, endometrial cancer, breast cancer, or liver cancer.

46. The method of claim 44, wherein the anti-cancer agent is a chemotherapeutic agent.

47. The method of claim 44, wherein the antibody is conjugated to a cytotoxin.

48. A kit comprising the antibody of claim 1.

49. The kit of claim 48 further comprising instructions for administering an effective amount of the antibody to an individual for treating nonhematopoietic cancer.

50. The kit of claim 48 further comprising instructions for administering an amount of the antibody and an amount of another anti-cancer agent to an individual for treating nonhematopoietic cancer, whereby the antibody and the anti-cancer agent in conjunction provide effective treatment of cancer in the individual.

51. A method of screening an antibody that specifically binds to a transferrin receptor expressed by nonhematopoietic cancer cells comprising the steps of a) providing multiple antibodies and selecting one or more antibodies that specifically bind to a transferrin receptor expressed by nonhematopoietic cancer cells and b) using the one or more antibodies selected from step a) to further select an antibody that does not specifically bind to a transferrin receptor expressed by activated T cells or by Jurkat cells.

52. The method of claim 51, wherein the antibody specifically binds to a carbohydrate on the transferrin receptor expressed by nonhematopoietic cancer cells.

53. The method of claim 51 further comprising the step of selecting the antibody that is capable of inducing apoptosis of the cancer cells after binding to transferrin receptor on cell surface of the cancer cells in the absence of cytotoxin conjugation and immune effector function.

54. The method of claim 51, wherein the nonhematopoietic cancer cells are pancreatic cancer cells, gastric cancer cells, colorectal cancer cells, lung cancer cells, ovarian cancer cells, prostate cancer cells, endometrial cancer cells, breast cancer cells, or liver cancer cells.


SEQUENCE LISTING (19 sequences)

SEQ ID NO: 1 - protein, 138 aa, Mus musculus
MEWSWIFLFLLSGTAGVLSEVELQQFGIEMVKPGASVKISCKASGYIFTDYHMDWVRQSHGKSLEWIGDIDPKYDRVTYNQKFKGKASLTADKSSSTAYMELRSLTSEDTAVYYCAKTGAYGDYLAYWGQGTLVTVSA

SEQ ID NO: 2 - DNA, 414 bp, Mus musculus
atggaatggagctggatctttctctttctcctgtcaggaactgcaggtgtcctctctgaggtcgagctgcaacagtttggaattgagatggtgaagcctggggcttcagtgaagatatcctgcaaggcctctggctacatattcactgactaccacatggactgggtgaggcagagccatggcaagagccttgagtggattggagatattgatcctaaatatgatagagttacctataaccagaagttcaagggaaaggcctcactgactgcagacaagtcctccagcacagcctacatggaactccgcagcctgacatctgaggacactgcagtctattactgtgcaaagacgggggcgtatggtgattatcttgcttactggggccaggggactctggtcactgtctccgca

SEQ ID NO: 3 - protein, 132 aa, Mus musculus
MKLPVRLLVLMFWIPASSSDLVMTQTPLSLPVSLGDQASISCRSSQSLVHSDGNTYFYWYLQKPGQSPKLLIYKVSNRFSGVPDRFSAGGSGTYFTLKISRVEAEDLGVYFCSQTTHFPPTFGGGTKLEIKR

SEQ ID NO: 4 - DNA, 396 bp, Mus musculus
atgaagttgcctgttaggctgttggtgctgatgttctggattcctgcttccagcagtgatcttgttatgacccaaactccactctccctgcctgtcagtcttggagatcaagcctccatctcttgcagatctagtcagagccttgtacacagtgatggaaacacctatttctattggtacctgcagaagccaggccagtctccaaagctcctgatctacaaagtttccaaccgattttctggggtcccagacaggttcagtgccggtggatcagggacatatttcacactcaagatcagcagagtggaggctgaggatctgggagtttatttctgctctcaaactacacattttcctccgacgttcggtggaggcaccaagctggaaatcaaacgg

SEQ ID NO: 5 - protein, 138 aa, Mus musculus
MGWSYIILFLVATATGVHSQVQLQQPGAELVKPGTSVKLSCKASGYNFTSYWINWVKLRPGQGLEWIGDIYPGSGSTNYNEKFKSKATLTVDTSSSTAYMQLSSLASEDSALYYCARSAYRYDWFAYWGQGTLVTVSA

SEQ ID NO: 6 - DNA, 414 bp, Mus musculus
atgggatggagctatatcatcctcttcttggtagcaacagctacaggtgtccactctcaggtccaactgcagcagcctggggctgagcttgtgaagcctgggacttcagtgaagctgtcctgcaaggcttctggctacaacttcaccagctactggataaactgggtgaagctgaggcctggacaaggccttgagtggattggagatatttatcctggtagtggtagtactaactacaatgagaagttcaagagcaaggccacactgactgtagacacatcctccagcacagcctacatgcaactcagcagcctggcatctgaggactctgctctctattactgtgcaagatccgcctataggtacgactggtttgcttactggggccaagggactctggtcactgtctctgca

SEQ ID NO: 7 - protein, 128 aa, Mus musculus
MSVPTQVLGLLLLWLTDARCDIQMTQSPASLSVSVGETVTITCRASENIYSNLAWYQQKQGKSPQLLVYAATNLADGVPSRFSGSGSGTQYSLKINSLQSEDFGSYYCQHFWGTPYTFGGGTKLEIKR

SEQ ID NO: 8 - DNA, 384 bp, Mus musculus
atgagtgtgcccactcaggtcctggggttgctgctgctgtggcttacagatgccagatgtgacatccagatgactcagtctccagcctccctatctgtatctgtgggagaaactgtcaccatcacatgtcgagcaagtgagaatatttacagtaatttagcatggtatcagcagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggagttattactgtcaacatttttggggtactccgtacacgttcggaggggggaccaagctggaaataaaacgg

SEQ ID NO: 9 - protein, 136 aa, Mus musculus
MLLGLKWVFFVVFYQGVHCEVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVAYGSRNYWGQGTTLTVSS

SEQ ID NO: 10 - DNA, 408 bp, Mus musculus
atgctgttggggctgaagtgggttttctttgttgttttttatcaaggtgtgcattgtgaggtgcagcttgttgagtctggtggaggattggtgcagcctaaagggtcattgaaactctcatgtgcagcctctggattcaccttcaatacctacgccatgaactgggtccgccaggctccaggaaagggtttggaatgggttgctcgcataagaagtaaaagtaataattatgcaacatattatgccgattcagtgaaagacaggttcaccatctccagagatgattcacaaagcatgctctatctgcaaatgaacaacttgaaaactgaggacacagccatgtattactgtgtggcttacggtagtagaaactactggggccaaggcaccactctcacagtctcctca

SEQ ID NO: 11 - protein, 134 aa, Mus musculus
MESQTQVLMSLLFWVSGTCGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPYTFGGGTKLEIKR

SEQ ID NO: 12 - DNA, 402 bp, Mus musculus
atggaatcacagactcaggtcctcatgtccctgctgttctgggtatctggtacctgtggggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgtacacgttcggaggggggaccaagctggaaataaaacgg

SEQ ID NO: 13 - protein, 138 aa, Mus musculus
MGWSWIFLFLLSGTAGVHSEVQLQQSGPEVVKPGASMKMSCKTSGYKFTGYYMDWVKQSLGASFEWIGRVIPSNGDTRYNQKFEGKATLTVDRSSSTAYMELNSLTSEDSAVYYCARKPLSGNAADYWGQGTSVTVST

SEQ ID NO: 14 - DNA, 414 bp, Mus musculus
atgggatggagctggatctttctcttcctcttgtcaggaactgcaggtgtccactctgaggtccagctgcaacagtctggacctgaggtggtgaagcctggggcttcaatgaagatgtcctgtaagacttctggatacaaattcactggctactacatggactgggtgaagcagagccttggagcaagctttgagtggattggacgtgttattccttccaatggtgatactaggtacaaccagaagttcgagggcaaggccacattgactgttgacaggtcctccagcacagcctacatggagctcaacagcctgacatctgaagactctgcggtctattactgtgcaagaaaaccgctaagtgggaatgctgcggactactggggtcaaggaacctcagtcaccgtctccaca

SEQ ID NO: 15 - protein, 130 aa, Mus musculus
MTMLSLAPLLSLLLLCVSDSRAETTVTQSPASLSVATGEKVTIRCITSTDIDDDMNWYQQKPGEPPKLLISDGNTLRPGVPSRFSSSGYGTDFVFTIENTLSEDITDYYCMQSDNMPFTFGSGTKLEIKR

SEQ ID NO: 16 - DNA, 390 bp, Mus musculus
atgaccatgctctcactagctcctctcctcagccttcttctcctctgtgtctctgattctagggcagaaacaactgtgacccagtctccagcatccctgtccgtggctacaggagagaaagtcactatcagatgcataactagcactgatattgatgatgatatgaactggtaccaacagaagccaggggaacctcctaaactccttatttcagatggcaatactcttcgtcctggagtcccatcccgattctccagcagtggctatggcacagattttgtttttacaattgaaaacacgctctcagaagatattacagattactactgtatgcagagtgataacatgccattcacgttcggctcggggacaaagttggaaataaaacgg

SEQ ID NO: 17 - protein, 449 aa, Artificial Sequence
  Synthetic Construct
EVQLQQSGPEVVKPGASMKMSCKTSGYKFTGYYMDWVKQSLGASFEWIGRVIPSNGDTRYNQKFEGKATLTVDRSSSTAYMELNSLTSEDSAVYYCARKPLSGNAADYWGQGTSVTVSTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, Artificial Sequence
  Synthetic Construct
ETTVTQSPASLSVATGEKVTIRCITSTDIDDDMNWYQQKPGEPPKLLISDGNTLRPGVPSRFSSSGYGTDFVFTIENTLSEDITDYYCMQSDNMPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF